(19) United States
(12) Patent Application Publication
(10) Pub. No.: US 2004/0175727 A1
(43) Pub. Date: Sep. 9, 2004

(54) Synthetic muscle promoters with activities exceeding naturally occurring regulatory sequences in cardiac cells

(75) Inventors: Ruxandra Draghia-Akli, Houston, TX (US); Robert J. Schwartz, Houston, TX (US)

Correspondence Address: T. Ling Chwang, Jackson Walker L.L.P., Suite 600, 2435 North Central Expressway, Richardson, TX, 75080, US

(73) Assignees: ADVISYS, Inc., The Woodlands, TX; Baylor College of Medicine, Houston, TX

(21) Appl. No.: 10/699,597

(22) Filed: Oct. 30, 2003

Related U.S. Application Data
(60) Provisional application No. 60/423,536, filed on Nov. 4, 2002.

(51) Int. Cl. C12Q 1/68; G01N 33/53; C12N 15/85

(52) U.S. Cl. 435/6; 435/7.1; 435/455

(57) ABSTRACT

Transgenes driven by naturally occurring cardiac promoters have relatively low levels of cardiac transgenic gene expression, and have consequently limited the use of cardiac muscle as a target for plasmid mediated gene supplementation. However, by randomly assembling motifs of E-box, MEF-2, TEF-1 and SRE elements, cardiac-specific synthetic promoter recombinant libraries have been produced. By screening hundreds of resultant clones for transcriptional activity both in vitro and in vivo, a few cardiac-specific synthetic promoters were discovered comprising a transcriptional potency that greatly exceeds the transcriptional levels obtained from natural myogenic and viral gene promoters. These promoters are used to direct the expression of desirable genes in nucleic acid expression constructs specifically to cardiac cells. Thus, these cardiac specific-synthetic promoters can be utilized for plasmid mediated gene supplementation for serious health conditions, such as ischemic disease, myocardial infarction or heart failure. Thus, one aspect of the current invention is a cardiac specific-synthetic promoter produced by a method that generates a library of randomized synthetic-promoter-recombinant expression constructs. Another aspect of the present invention is directed to a method using the cardiac specific-synthetic expression construct for expression a gene of interest in a cardiac cell.

This application claims priority to U.S. Provisional Patent Application, Serial Number 60/423,536, entitled “Synthetic Muscle Promoters with Activities Exceeding Naturally Occurring Regulatory Sequences in Cardiac Cells,” filed on Nov. 4,2002, the entire content of which is hereby incorporated by reference.

### BACKGROUND

The ability to program recombinant gene expression in cardiac myocytes in vitro and vivo holds promise for the treatment of many inherited and acquired cardiovascular diseases (Lin et al., 1990). Cardiac and skeletal muscles are attractive targets for plasmid mediated gene supplementation because of their long life span, and large capacity for protein synthesis and secretion for local or general effects (Draghia-Akli et al., 1999; Vale et al., 1999). Moreover cardiac and skeletal muscle tissue is highly vascularized and has a high rate of blood flow, thus allowing de novo proteins to readily act locally or enter the systemic circulation. Importantly, direct administration of plasmid DNA into heart or muscle leads to expression of recombinant proteins in muscle cells (Mazda, 2002; Prentice et al., 1996). Plasmid DNA can persist in an episomal state directing the expression of recombinant proteins for months to years (Acsadi et al., 1991; Wolff et al., 1992). However, a limiting problem in using plasmid mediated gene supplementation to correct or prevent cardiac disease has been the relatively low levels of expression that have been achieved with muscle specific vectors. Although a low expression level of a recombinant protein is enough to generate an immune response against the expressed protein, therapeutic levels of recombinant proteins have currently not been produced using muscle specific promoters/enhancers (Montgomery et al., 1997). In this report we describe a method for the construction and characterization of synthetic promoters for cardiac and muscle tissue. The transcriptional potency of these synthetic promoters in terminally differentiated muscle greatly exceeds that of the natural myogenic skeletal α-actin gene promoter and viral promoters.

When delivering therapeutic genes, the use of tissue specific promoters is highly desirable. Numerous strategies have been employed to create or use for therapeutic purposes tissue specific promoters, which support transcription in cardiac and skeletal muscle, and are essentially silent in other cell types (Keogh et al., 1999; Roell et al., 2002; Rothermel et al., 2001). This approach assures localized transgene activity, without the potential complication of side effects linked to inappropriate expression in non-target tissues or organs. For instance, because of safety issues, components of the beta-adrenergic, Akt or caspase signaling pathway cannot currently be viewed as attractive targets for human gene therapy. Rather, the balance of evidence supports strategies that will target gene products specifically and directly at cardiac regulation, and molecular techniques can be devised to modulate their activity specifically and conditionally (Condorelli et al., 2001; Ding et al., 2002; Webster and Bishopric, 2000). In this report we describe a method for the construction and characterization of synthetic promoters for cardiac and muscle tissue. The transcriptional potency of these synthetic promoters in terminally differentiated muscle greatly exceeds that of the natural myogenic skeletal α-actin gene promoter and viral promoters, and may have important applications in conjunction with therapeutic genes.

Analysis of the organization of several strong muscle promoters and enhancers, with respect to groupings of cis-acting regulatory elements and their interactions with myogenic regulatory factors led the inventors to formulate a strategy to construct synthetic muscle promoters. Myogenic restricted promoters, such as those of the α-actins, display complex organization. Activation often requires interactions of various myogenic trans-factors with pairs of specific cis-elements. These elements are evolutionarily conserved and primarily responsible for tissue specific expression in adult skeletal muscle, and appeared to be a logical choice for generating synthetic promoters. By randomly assembling these myogenic elements into synthetic promoter (“SP”) recombinant libraries, and then by screening hundreds of the resultant clones for transcriptional activity it was possible to create artificial promoters whose transcriptional potency exceeds that of any naturally occurring promoters, as described in U.S. Pat. No. 6,410,228 (“the '228 Patent), issued on Jun. 25, 2002 and entitled “Method for the Identification of Synthetic Cell- or Tissue Specific Transcriptional Regulatory Regions” with Schwartz et al., listed as inventors, the entire content of which is hereby incorporated by reference.

The molecular mechanisms controlling cardiac-specific gene transcription requires the dissection of the cis-elements that govern the complex spatio-temporal expression of these genes. The vertebrate heart is formed during fetal development following a series of complex morphogenetic events that require the functional presence of different proteins, tightly regulated by combinatorial interactions of several transcription factors and their cofactors (Nemer and Nemer, 2001; Wang et al., 2001). First, the proximal serum response element (SRE) (′5-CC[A/T]₆GG-3′) of the skeletal α-actin promoter was incorporated. Multiple SREs are found in the cardiac, skeletal and smooth muscle α-actin promoters (Chang et al., 2001), and in the promoters of myosin light chain and dystrophin (Bergsma et al., 1986; Carroll et al., 1986). This cis-element is recognized by the trans-acting serum response factor (SRF), and by the competitive inhibitor YY1 (Chow and Schwartz, 1990; Lee et al., 1992; Minty and Kedes, 1986). Serum response factor (SRF) is a key regulator of a number of extracellular signal-regulated genes important for cell growth and differentiation (Zhang et al., 2001). Mutations in the proximal SRE that block SRF binding abolish skeletal α-actin promoter (SK) activity, indicating a fundamental role for this promoter element. Second, MEF-2 sites (′5-[C/T]TAAAAATAAC[C/T]₃-3′) that have been found in the promoter/enhancer regions of the myosin light-chain 3 gene were selected. A single MEF-2 site lacks enhancer activity, but has multiple copies that exhibit strong enhancer activity (Gossett et al., 1989). Mutation of the MEF2 site severely reduced promoter activity in embryos, underlining the importance of MEF2 in controlling differentiation in all muscle lineages (Kelly et al., 2002). Third, the MEF-1 sites (′5-CANNTG-3′), or E-boxes that are found in the upstream regulatory region of most, if not all, muscle-specific genes were included (Olson et al., 1991; Weintraub et al., 1990). MEF-1 sites are recognized by the basic helix-loop-helix (bHLH) family of proteins. Multiple MEF-1 sites placed upstream of basal non-muscle promoters are sufficient to direct muscle-specific expression and MyoD-mediated trans-activation in transient assays (Lassar et al., 1991; Weintraub et al., 1990). Finally, the highly conserved muscle-CAT motif, or TEF-1 binding site (′5-CATTCCT-3′) was selected. TEF-1 mediates both muscle-specific (SK, cardiac troponin T, cardiac α- and β-myosin heavy chain) and non-muscle specific transcription (simian virus 40 promoter) (Larkin et al., 1996; Stewart et al., 1994).

In M-CAT dependent promoters, specific sequences immediately flanking the core motif contribute to both the cell specificity and the overall transcriptional strength (O'Connell et al., 2001). While the process of creating synthetic promoters and their muscle specificity have been previously described by us (Li et al., 1999), their cardiac specificity has not been described or demonstrated. For instance, unpublished data from our laboratory proved that the skeletal α-actin 448 (SK448) is expressed exclusively in the skeletal muscle in transgenic animals, while the longer version of the same promoter, skeletal α-actin 622 (SK622) is expressed both in the skeletal muscle and in the cardiac muscle. Also, data from transgenic animals, an artificial model, cannot be extrapolated to direct transfection or in vivo activity after direct injection. For instance, after direct injection or in vitro cell transfection, the skeletal α-actin 448 (SK448) is expressed in cardiac cells.

Transgenes driven by naturally occurring cardiac promoters have relatively low levels of cardiac transgenic gene expression, and have consequently limited the use of cardiac muscle as a target for plasmid mediated gene supplementation. However, by randomly assembling motifs of E-box, MEF-2, TEF-1 and SRE elements, cardiac-specific synthetic promoter recombinant libraries have been produced. By screening hundreds of resultant clones for transcriptional activity both in vitro and in vivo, a few cardiac-specific synthetic promoters were discovered comprising a transcriptional potency that greatly exceeds the transcriptional levels obtained from natural myogenic and viral gene promoters. These promoters are used to direct the expression of desirable genes in nucleic acid expression constructs specifically to cardiac cells. Thus, these cardiac specific-synthetic promoters are further utilized during plasmid mediated gene supplementation for serious health conditions, such as ischemic disease, myocardial infarction or heart failure. Thus, one aspect of the current invention is a cardiac specific-synthetic promoter produced by a method that generates a library of randomized synthetic-promoter-recombinant expression constructs. A second aspect of the present invention is directed to a method using the cardiac specific-synthetic expression construct for expression a gene of interest in a cardiac cell.

### SUMMARY

A first aspect of the current invention comprises a cardiac specific-synthetic promoter. This promoter is produced by a method comprising the steps of: introducing a library of randomized synthetic-promoter-recombinant expression constructs into a first-population of cells forming a first-test-population of cells; screening the first-test-population of cells for a first cardiac-specific-clone having a first-transcriptional activity that is higher than a control-transcriptional activity; and utilizing the cardiac specific-synthetic promoter from the first-cardiac-specific clone as the cardiac specific-synthetic promoter for a cardiac-specific-synthetic expression construct. In this way, each of the randomized synthetic-promoter-recombinant expression constructs are operatively linked to a reporter gene to form a nucleic acid expression construct; and the control-cardiac-specific-clone comprises a known-promoter operatively linked to the reporter gene, which forms a control-nucleic acid expression construct having the control-transcriptional activity in the first-population of cells. One specific embodiment of the current invention further comprises a second-screening the first cardiac-specific-clone in a second-test-population of cells before utilizing the cardiac-specific-synthetic promoter as the cardiac-specific-synthetic promoter for the cardiac-specific-synthetic expression construct. When the second-screening is performed, the reporter gene from the first-cardiac-specific-clone has a second-transcriptional activity in the second-population of cells that is higher than a second-control-transcriptional activity of the control-cardiac-specific-clone introduced into the second-population of cells. Additionally, the first-population of cells comprise cells in vitro, and the second-population of cells comprise cells in vivo. In a specific embodiment of this invention, the cardiac specific synthetic promoter comprises c5-12 (SeqID#5). Other specific embodiment utilizes other cardiac specific synthetic promoters such as c1-26 (SeqID#16); c2-26 (SeqID#17); c2-27 (SeqID#18); c5-5 (SeqID#19); c6-5 (SeqID#20); c6-16 (SeqID#21); or c6-39 (SeqID#22). The cardiac-specific-synthetic promoters comprise a first-combination of cis-acting regulatory elements, and the first combination of cis-acting regulatory elements were selected from a library of randomized synthetic-promoter-recombinants. The cardiac-specific synthetic promoter drives a transcriptional activity of the expressible gene in a population of cells that is higher than the transcriptional activity of the expressible gene driven by a control-promoter in the same population of cells. The cis-acting regulatory elements utilized for the cardiac-specific synthetic promoter comprise SRE (SeqID#1); MEF-1 (SeqID#2); MEF-2 (SeqID#3); and TEF-1 (SeqID#4).

A second aspect of the current invention is a method for using a cardiac specific-synthetic expression construct for expressing a gene in a cardiac cell. The method comprises delivering into the cardiac cell, a cardiac specific-synthetic expression construct. The cardiac-specific-synthetic expression construct comprises a cardiac-specific-synthetic-promoter operatively-linked to an expressible gene. In a specific embodiment of this invention, the cardiac specific synthetic promoter comprises c5-12 (SeqID#5). Other specific embodiment utilizes other cardiac specific synthetic promoters such as c1-26 (SeqID#16); c2-26 (SeqID#17); c2-27 (SeqID#18); c5-5 (SeqID#19); c6-5 (SeqID#20); c6-16 (SeqID#21); or c6-39 (SeqID#22). The cardiac-specific-synthetic promoters comprise a first-combination of cis-acting regulatory elements, and the first combination of cis-acting regulatory elements were selected from a library of randomized synthetic-promoter-recombinants. The cardiac-specific synthetic promoter drives a transcriptional activity of the expressible gene in a population of cells that is higher than the transcriptional activity of the expressible gene driven by a control-promoter in the same population of cells. The cis-acting regulatory elements utilized for the cardiac-specific synthetic promoter comprise SRE (SeqID#1); MEF-1 (SeqID#2); MEF-2 (SeqID#3); and TEF-1 (SeqID#4). Certain embodiments describe the expressible-gene comprising a nucleic acid sequence that encodes a growth-hormone-releasing-hormone (“GHRH”) or functional biological equivalent thereof. The encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide. In another specific embodiment, the encoded GHRH or functional biological equivalent thereof is of formula (SEQID#6): The cardiac specific-synthetic expression constructs of this invention also comprises SeqID No: 7, SeqID No: 8, SeqID No: 9, SeqID No: 10, SeqID No: 11, SeqID No: 12, SeqID No: 13, SeqID No: 14, or SeqID No: 15.

### BRIEF DESCRIPTION OF FIGURES

FIG. 1 shows the strategy and design of muscle synthetic promoters with the proportion of regulatory elements in different combinations of synthetic promoters, wherein each combination contains at least one of each muscle specific regulatory elements;

FIG. 2 shows the design of muscle synthetic promoters elements in the constructs with the highest in vitro reporter gene activity compared with skeletal α-actin 448 promoter (“SK448”);

FIG. 3 shows the transcriptional expression of luciferase in fold excess of the SK448 expression, the luciferase reporter gene was driven by the various synthetic promoters and activity was measured at 48 hours post-differentiation;

FIG. 4 shows the transcriptional expression of luciferase in anterior tibialis of adult ICR mice driven by the synthetic promoters SPc1-28, SPc5-12, cytomegalovirus (“CMV”); and SK448, the luciferase activity was measured at 7 days after direct injections in anterior tibialis;

FIG. 5 shows the transcriptional expression of β-galactosidase (“β-gal”) in primary chicken muscle culture driven by the synthetic promoters cytomegalovirus (“CMV”), SK448, SPc5-12, and control, the β-gal activity was measured at 24, 48, 72, and 96 hours;

FIG. 6 shows the transcriptional expression of luciferase in primary mouse cardiac culture driven by the synthetic promoters cytomegalovirus (“CMV”), SPc5-12, SK448, SV40, β-gal, and non-transfected cells the luciferase activity was measured at 24,48, 72, and 96 hours;

FIG. 7 shows a time course table for Beta-galactosidase activity in cardiac myocytes wherein the activity of β-gal was measured at 24, 48, 72, and 96 hours;

FIG. 8 shows the in vitro muscle specific expression of β-gal driven by the synthetic promoter SPc5-12, wherein the expression level of β-gal driven by SPc5-12 promoter is comparable with the expression level of β-gal driven by the SK448 promoter in displaying cell type specific expression, and the expression level of β-gal driven by SPc5-12 promoter is at least one order of magnitude less active then the β-gal driven by the CMV promoter in several non-muscle cell lines (CV1, 293, HeLa and 10T1/2);

FIG. 9 shows the expression level of β-gal driven by the synthetic promoter c5-12 is muscle and cardiac specific in vivo, a total RNA Northern blot of various tissues (e.g. testis (“T”), brain (“B”), intestine (“I”), lung (“Lg”), stomach (“St”), kidney (“K”), liver (“Lv”), gastrocnemius (“M”), heart (“H”), spleen (“Sp”)) from different lines of transgenic mice hybridized with a β-gal cDNA probe and then a mouse 18S probe, was used to show the muscle and cardiac specific expression of a reporter gene driven by SPc5-12;

FIG. 10 shows the in vivo expression of a luciferase reporter gene driven by the synthetic promoters cytomegalovirus (“CMV”), SPc5-12, SK448, SV40, and control, wherein the in vivo luciferase activity was analyzed at 2 and 4 weeks after direct intra-muscular injection;

FIG. 11 shows the level of mouse growth hormone (“GH”) in mice that were injected with a GHRH expression construct driven by the SPc5-12 promoter when compared with control promoters, the GH levels were determined at 7 days post-injection;

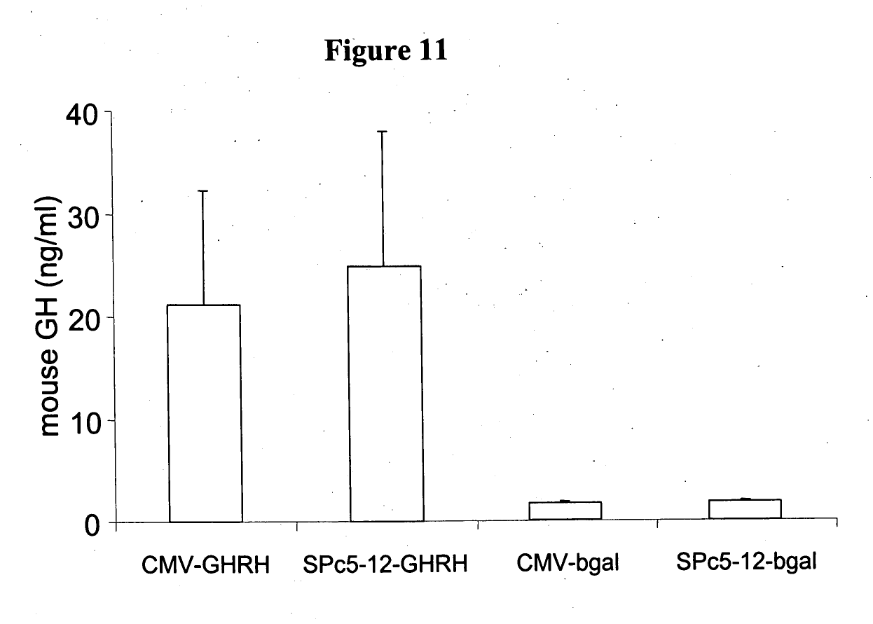

FIG. 12 shows the synthetic promoter c1-26 sequence with the regulatory elements marked and with the restriction maps;

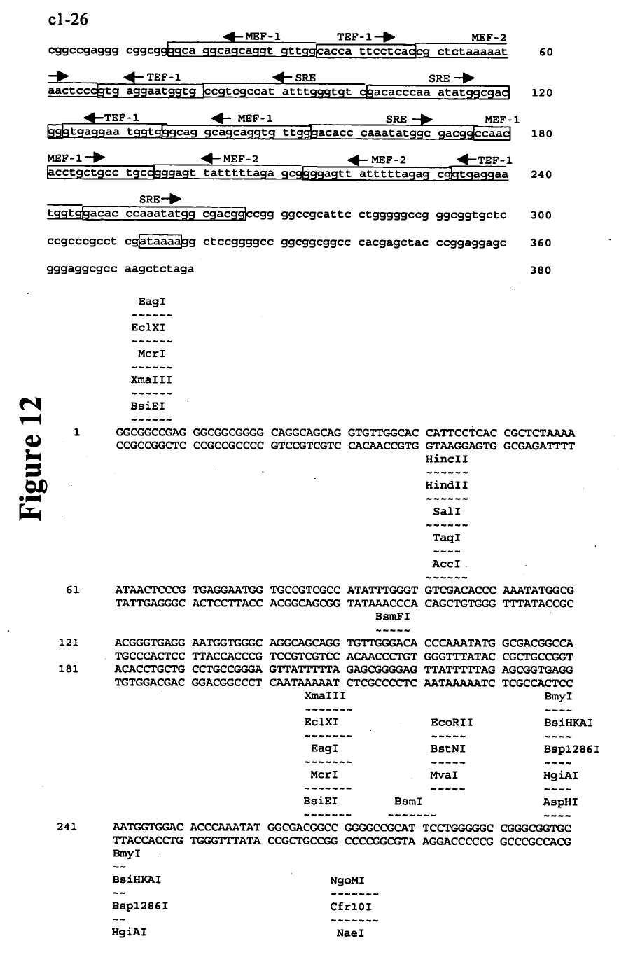

FIG. 13 shows the synthetic promoter c2-26 sequence with the regulatory elements marked and with the restriction maps;

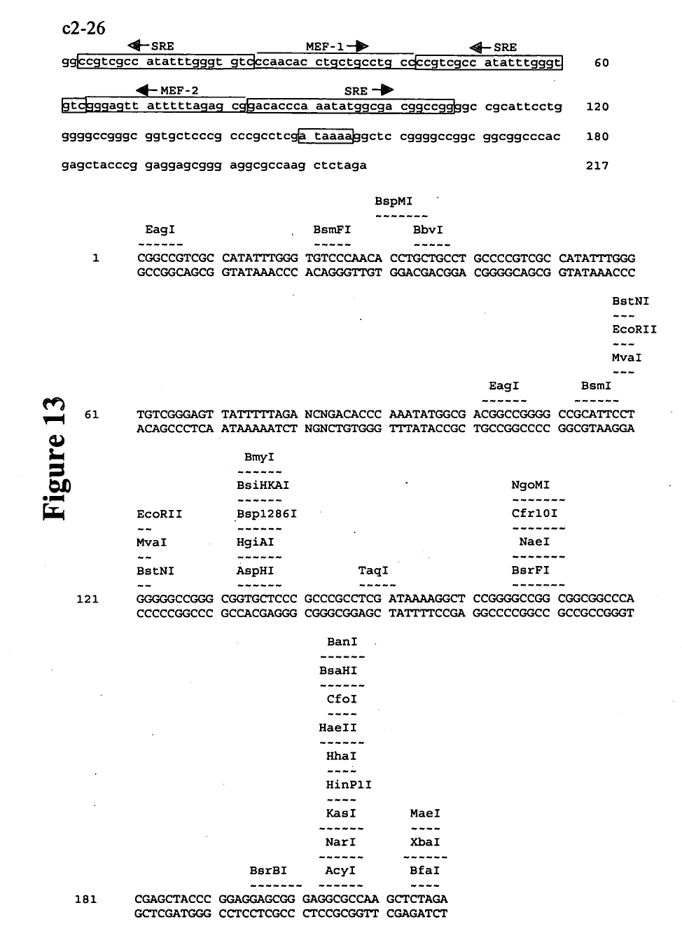

FIG. 14 shows the synthetic promoter c2-27 sequence with the regulatory elements marked and with the restriction maps;

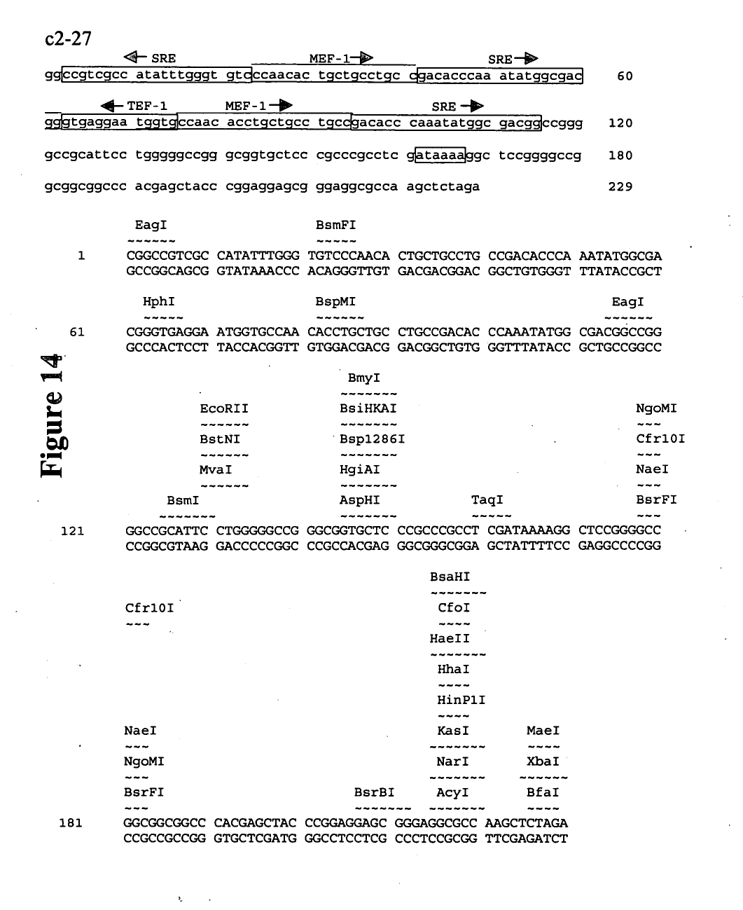

FIG. 15 shows the synthetic promoter c5-5 sequence with the regulatory elements marked and with the restriction maps;

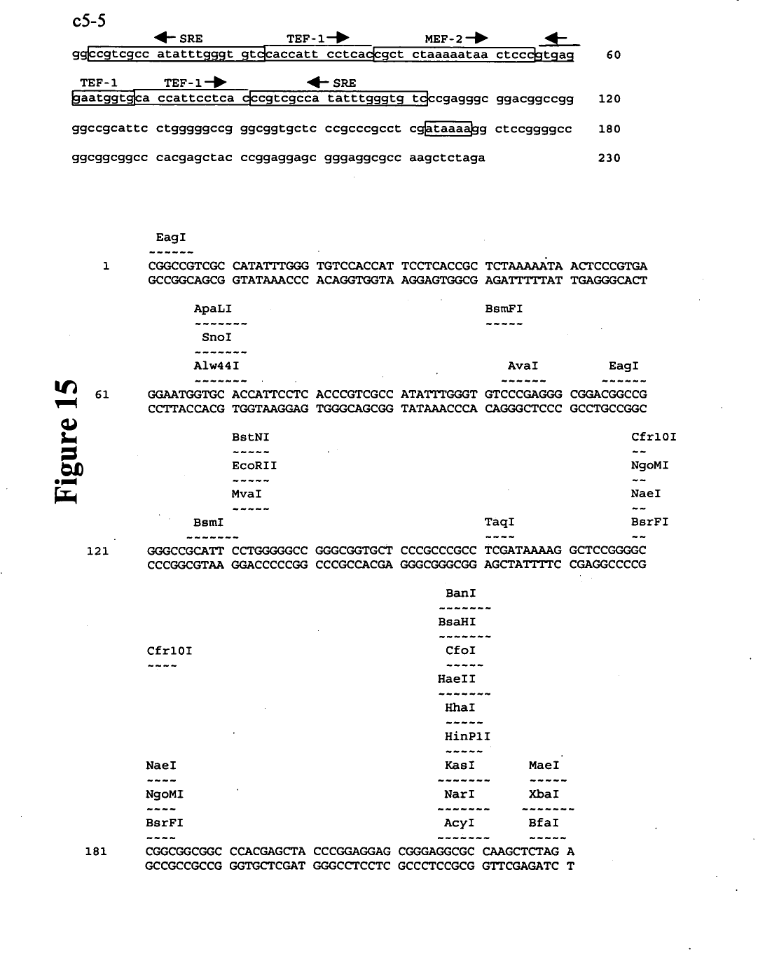

FIG. 16 shows the synthetic promoter c5-12 sequence with the regulatory elements marked and with the restriction maps;

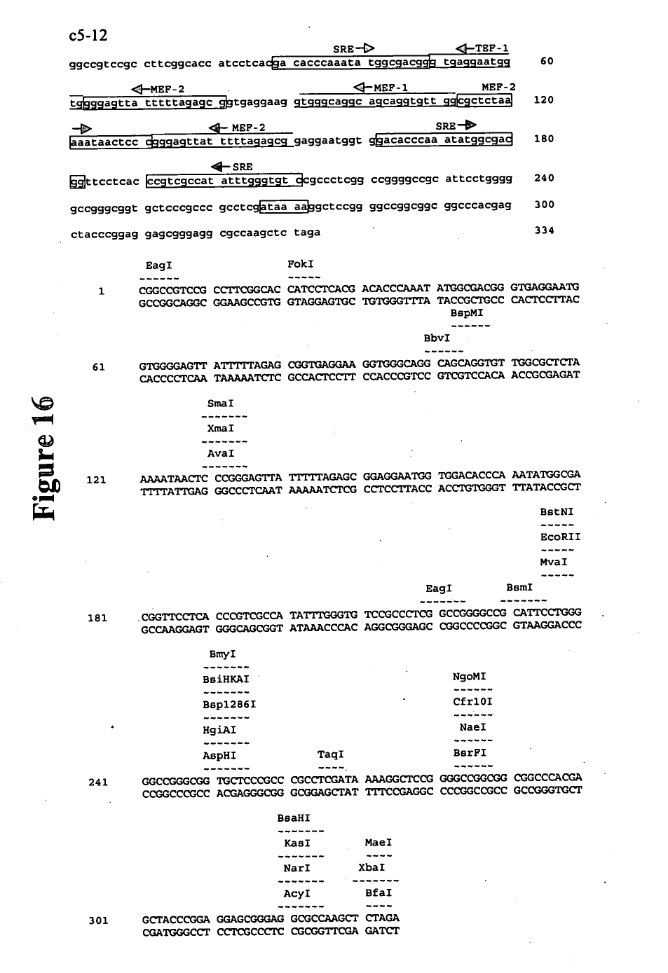

FIG. 17 shows the synthetic promoter c6-5 sequence with the regulatory elements marked and with the restriction maps;

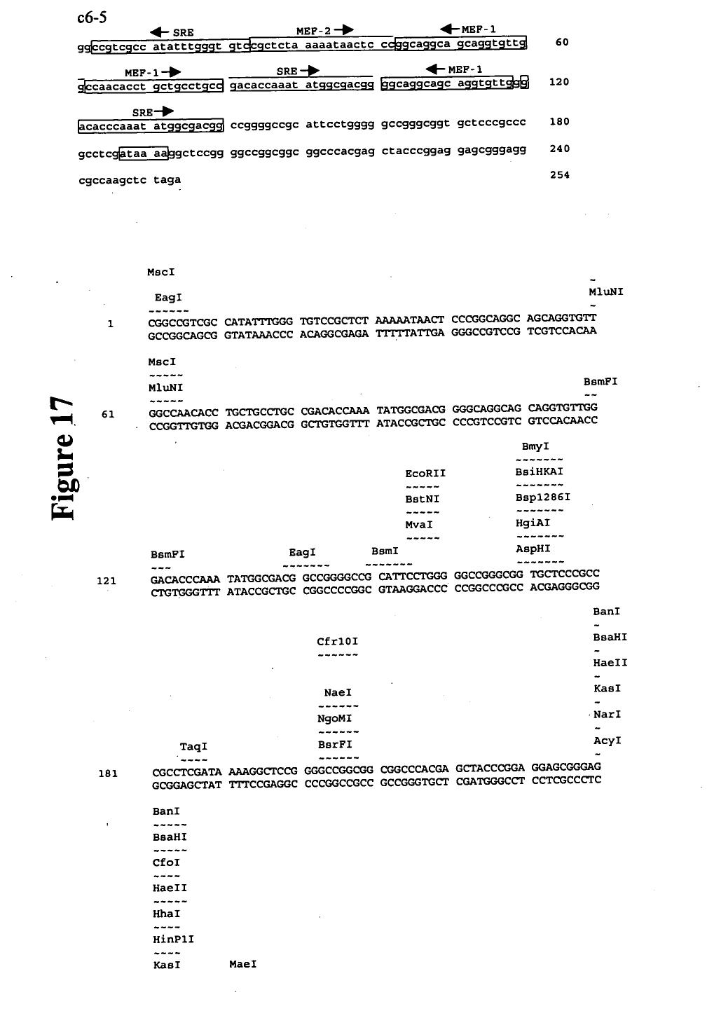

FIG. 18 shows the synthetic promoter c6-16 sequence with the regulatory elements marked and with the restriction maps;

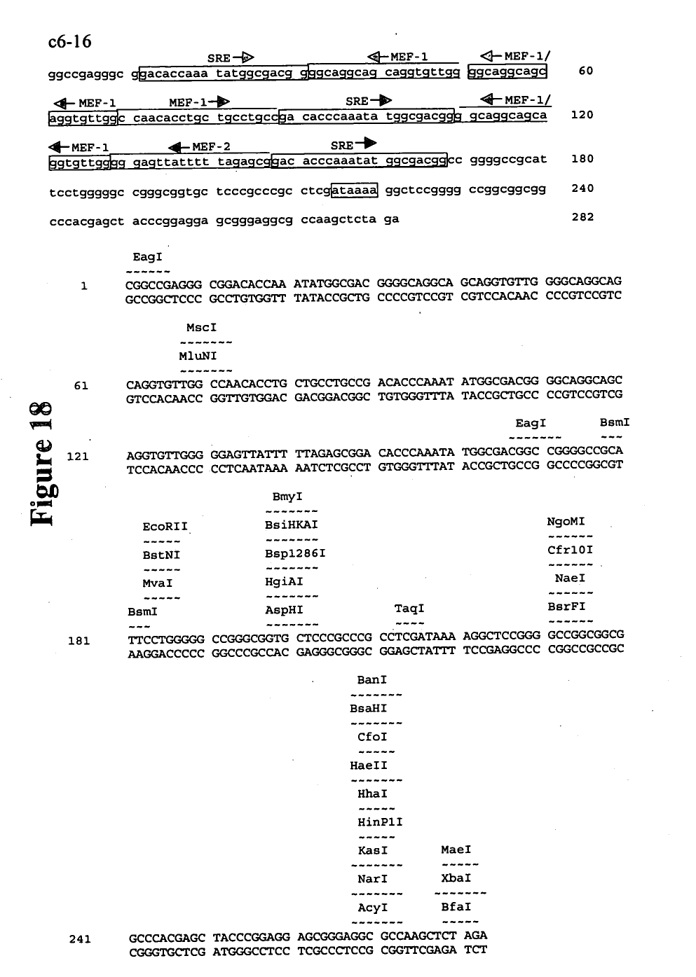

FIG. 19 shows the synthetic promoter c6-39 sequence with the regulatory elements marked and with the restriction maps.

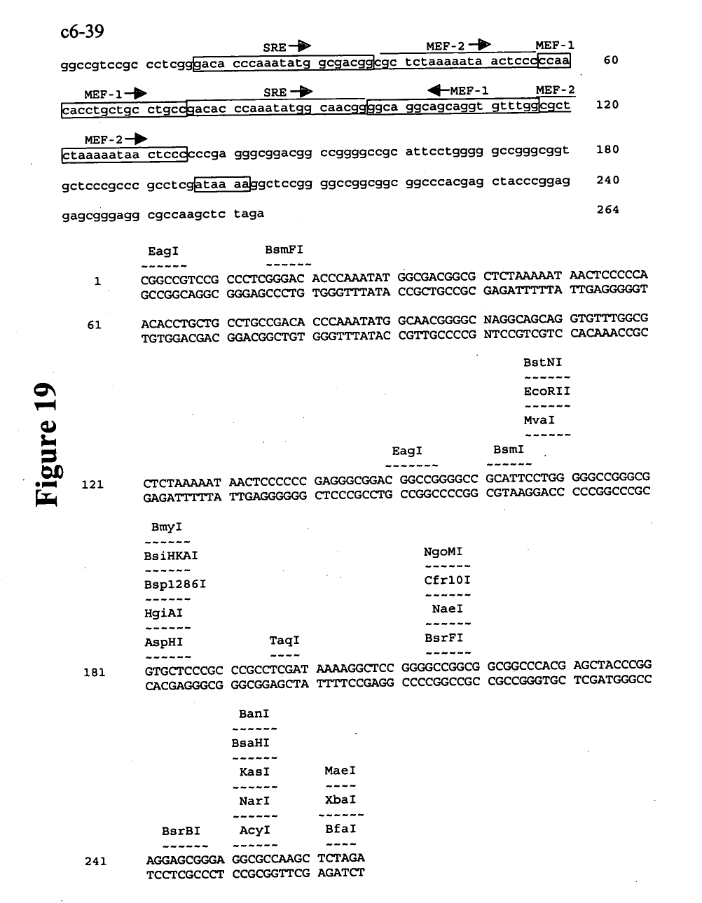

### DETAILED DESCRIPTION OF PREFERRED EMBODIMENTS

Terms:

The term “a” or “an” as used herein in the specification may mean one or more. As used herein in the claim(s), and when used in conjunction with the word “comprising”, the words “a” or “an” may mean one or more than one. As used herein “another”may mean at least a second or more.

The term “cis-acting regulatory elements” as used herein refers nucleic acid sequences that comprise transcription factor binding sites. In specific embodiments, the cis-acting regulatory elements comprise the muscle-specific control elements SRE, MEF-1, MEF-2, and TEF-1. It is recognized by one of ordinary skill in the art that other control elements may also be utilized in the present invention.

The term “operatively linked” as used herein refers to elements or structures in a nucleic acid sequence that are linked by operative ability and not physical location. The elements or structures are capable of, or characterized by accomplishing a desired operation. It is recognized by one of ordinary skill in the art that it is not necessary for elements or structures in a nucleic acid sequence to be in a tandem or adjacent order to be operatively linked.

The term “plasmid” as used herein refers generally to a construction comprised of extra-chromosomal genetic material, usually of a circular duplex of DNA that can replicate independently of chromosomal DNA. Plasmids, or fragments thereof, may be used as vectors. Plasmids are double-stranded DNA molecule that occur or are derived from bacteria and (rarely) other microorganisms. However, mitochondrial and chloroplast DNA, yeast killer and other cases are commonly excluded.

The term “plasmid mediated gene supplementation” as used herein refers a method to allow a subject to have prolonged exposure to a therapeutic range of a therapeutic protein by utilizing a nucleic acid expression construct in vivo.

The term “promoter” as used herein refers to a sequence of DNA that directs the transcription of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a “tissue-specific” or “tissue-preferred” manner, such that it is only active in transcribing the operable linked coding region in a specific tissue type or types. Additionally, promoters may comprise “viral promoters,” “control-promoters,” “naturally-occurring,” or “synthetically” assembled nucleic acid sequences.

The term “randomized synthetic-promoter-recombinants” as used herein are assembled combinations of randomized cis-acting regulatory elements.

The term “reporter gene” as used herein are nucleic acid sequences encoding easily assayed proteins. They are used to replace other coding regions whose protein products are difficult to assay. Among the more commonly used reporter genes are those for the following proteins chloramphenicol acetyltransferase (“CAT”), β-galactosidase (“GAL”), β-glucuronidase (“GUS”), luciferase (“LUC”), and green fluorescent protein (“GFP”). It is recognized by one of ordinary skill in the art that other reporter genes are available. It is also recognized by one of ordinary skill in the art that other coding regions (e.g. therapeutic genes) are easily substituted in lieu of the reporter gene.

The term “transcriptional activity” as used herein refers to the transcription of the information encoded in DNA into a molecule of a RNA, or the translation of the information encoded in the nucleotides of a RNA molecule into a defined sequence of amino acids in a protein.

The term “vector” as used herein refers to any vehicle that delivers a nucleic acid into a cell or organism. Examples include plasmid vectors, viral vectors, liposomes, or cationic lipids. The term “vector” as used herein more specifically refers to a construction comprised of genetic material designed to direct transformation of a targeted cell by delivering a nucleic acid sequence into that cell. A vector may contain multiple genetic elements positionally and sequentially oriented with other necessary elements such that an included nucleic acid cassette can be transcribed and when necessary translated in the transfected cells. These elements are operatively linked. The term “expression vector” refers to a DNA plasmid that contains all of the information necessary to produce a recombinant protein in a heterologous cell.

A first aspect of the current invention comprises a cardiac specific-synthetic promoter. This promoter is produced by a method comprising the steps of: introducing a library of randomized synthetic-promoter-recombinant expression constructs into a first-population of cells forming a first-test-population of cells; screening the first-test-population of cells for a first cardiac-specific-clone having a first-transcriptional activity that is higher than a control-transcriptional activity; and utilizing the cardiac specific-synthetic promoter from the first-cardiac-specific clone as the cardiac specific-synthetic promoter for a cardiac-specific-synthetic expression construct. In this way, each of the randomized synthetic-promoter-recombinant expression constructs are operatively linked to a reporter gene to form a nucleic acid expression construct; and the control-cardiac-specific-clone comprises a known-promoter operatively linked to the reporter gene, which forms a control-nucleic acid expression construct having the control-transcriptional activity in the first-population of cells. One specific embodiment of the current invention further comprises a second-screening the first cardiac-specific-clone in a second-test-population of cells before utilizing the cardiac-specific-synthetic promoter as the cardiac-specific-synthetic promoter for the cardiac-specific-synthetic expression construct. When the second-screening is performed, the reporter gene from the first-cardiac-specific-clone has a second-transcriptional activity in the second-population of cells that is higher than a second-control-transcriptional activity of the control-cardiac-specific-clone introduced into the second-population of cells. Additionally, the first-population of cells comprise cells in vitro, and the second-population of cells comprise cells in vivo. In a specific embodiment of this invention, the cardiac specific synthetic promoter comprises c5-12 (SeqID#5). Other specific embodiment utilizes other cardiac specific synthetic promoters such as c1-26 (SeqID#16); c2-26 (SeqID#17); c2-27 (SeqID#18); c5-5 (SeqID#19); c6-5 (SeqID#20); c6-16 (SeqID#21); or c6-c39 (SeqID#22). The cardiac-specific-synthetic promoters comprise a first-combination of cis-acting regulatory elements, and the first combination of cis-acting regulatory elements were selected from a library of randomized synthetic-promoter-recombinants. The cardiac-specific synthetic promoter drives a transcriptional activity of the expressible gene in a population of cells that is higher than the transcriptional activity of the expressible gene driven by a control-promoter in the same population of cells. The cis-acting regulatory elements utilized for the cardiac-specific synthetic promoter comprise SRE (SeqID#1); MEF-1 (SeqID#2); MEF-2 (SeqID#3); and TEF-1 (SeqID#4).

A second aspect of the current invention is a method for using a cardiac specific-synthetic expression construct for expressing a gene in a cardiac cell. The method comprises delivering into the cardiac cell, a cardiac specific-synthetic expression construct. The cardiac-specific-synthetic expression construct comprises a cardiac-specific-synthetic-promoter operatively-linked to an expressible gene. In a specific embodiment of this invention, the cardiac specific synthetic promoter comprises c5-12 (SeqID#5). Other specific embodiment utilizes other cardiac specific synthetic promoters such as c1-26 (SeqID#16); c2-26 (SeqID#17); c2-27 (SeqID#18); c5-5 (SeqID#19); c6-5 (SeqID#20) c6-39 (SeqID#22). The cardiac-specific-synthetic promoters comprise a first-combination of cis-acting regulatory elements, and the first combination of cis-acting regulatory elements were selected from a library of randomized synthetic-promoter-recombinants. The cardiac-specific synthetic promoter drives a transcriptional activity of the expressible gene in a population of cells that is higher than the transcriptional activity of the expressible gene driven by a control-promoter in the same population of cells. The cis-acting regulatory elements utilized for the cardiac-specific synthetic promoter comprise SRE (SeqID#1); MEF-1 (SeqID#2); MEF-2 (SeqID#3); and TEF-1 (SeqID#4). Certain embodiments describe the expressible-gene comprising a nucleic acid sequence that encodes a growth-hormone-releasing-hormone (“GHRH”) or functional biological equivalent thereof. The encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide. In another specific embodiment, the encoded GHRH or functional biological equivalent thereof is of formula (SEQID#6): The cardiac specific-synthetic expression constructs of this invention also comprises SeqID No: 7, SeqID No: 8, SeqID No: 9, SeqID No: 10, SeqID No: 11, SeqID No: 12, SeqID No: 13, SeqID No: 14, or SeqID No: 15.

The randomized synthetic-promoter-recombinants of this invention are prepared by a method comprising: identifying pools of cis-acting regulatory elements; and assembling the cis-acting regulatory elements in a random order to form the library of the synthetic-promoter-recombinants. The cis-acting regulatory elements comprise a double stranded, phosphorylated core motif that is flanked by an adjacent sequence. The assembled cis-acting regulatory elements face a same side of a DNA helix in each recombinant comprising the synthetic-promoter-recombinant library. The tissue specific synthetic promoter comprises a muscle specificity, wherein the muscle specificity comprises cardiac or skeletal muscle. A specific synthetic promoter of this invention comprises about 5 to about 20 cis-acting regulatory elements, wherein the regulatory elements comprise SRE (SeqID#1); MEF-1 (SeqID#2); MEF-2 (SeqID#3); and TEF-1 (SeqID#4). One example of a tissue specific synthetic promoter comprise SeqID#5. Additionally, the tissue specific synthetic promoter is utilized for plasmid mediated gene supplementation.

The regulatory regions of most promoters and enhancers consist of a combination of multiple transcription factor binding sites. Although not wanting to be bound by theory, the composition and arrangement of the binding sites determine the characteristics of regulatory regions. Expression vectors have been frequently modified by combining naturally existing promoters and enhancers (Hartikka et al., 1996; Skarli et al., 1998), and generally these modifications had little or no effect when compared with the transcriptional activity of the native promoters (Franz et al., 1997). In addition, naturally occurring regulatory regions are not always capable of regulating transcription in a desired manner (e.g. enhanced tissue specific regulation). In the invention described herein utilize specific transcription factor binding elements that were incorporated into synthetic promoters. For example, the muscle-specific control elements SRE, MEF-1, MEF-2, TEF-1 were synthesized, randomly assembled, and screened. Fragments containing 5-20 control elements represent synthetic promoter/enhancers were randomly ligated with regulatory sequences that varied in content, location and orientation relative to natural muscle promoters. These fragments were cloned in reporter plasmids in order to identify synthetic promoters with high transcriptional activity both in vitro and in vivo. Over 1000 different clones were evaluated. Since the method to produce and identify synthetic promoters with high transcriptional activity in vitro and in vivo is highly dependent on specific control elements and screening methods, it could not have been predicted by one skilled in the art which elements and control elements were appropriate without laborious and failed experimentations. However, the preferred composition and methods that are outlined for this invention achieve the desired in vitro and in vivo transcriptional activity.

We observed that multimerized single elements had low activity when compared with the natural skeletal α-actin 448 promoter (SK448), while about 2.5% of the clones derived by combining regulatory elements in the promoter library revealed 2-10 fold higher activity. Transfection assays in primary chicken myotubes indicated that one of the specific promoters of this invention (i.e. SPc5-12) had a 6 fold increased activity over a CMV promoter and at least 10 fold greater activity than the control SK448 promoter.

Transfection assays in primary mouse cardiac cells indicated that SPc5-12 had a 2 fold increased activity over CMV promoter and at least 13 fold greater activity than SK448. Analysis of direct intramuscular injection of DNA plasmids in normal muscle after 2-4 weeks revealed a 3-4 fold increased activity of SPc5-12 over SK448 promoter and a 6-8 fold increase over the CMV promoter. Cardiac and muscle specificity was confirmed with non-muscle cell lines and in transgenic animals.

Although not wanting to be bound by theory, many transcriptional regulatory regions have been described without the cloning of the corresponding transcription factors. Consequently, these “potential transcription factor regulatory elements” still need to be confirmed as functional for transcription regulation by identification of the corresponding transcription factors. Thus, synthetic promoters of this invention are constructed using combinations of cis-elements whose trans-factors are both known and unknown. Synthetic promoter libraries are also utilized to provide the basis for a desired functional tissue specificity. Because PCR mutagenesis allows the random modification of regulatory regions, this PCR method is utilized to screen even a greater number of regulatory regions by selection assays.

Although not wanting to be bound by theory, this novel system of designing synthetic promoters/enhancers using individual regulatory elements rather than entire promoters represents a significant improvement over previously generated plasmid DNA expression vectors (Buvoli et al., 2002; Phillips et al., 2002; Xu et al., 2002). For example, organ-specific promoter/enhancer fragments that exhibit persistent and increased expression when compared to naturally occurring sequences were obtained using this novel strategy. Although in vitro assays provide a good indication of promoter potency, in vivo studies are still required to determine the most appropriate synthetic promoter, as indicated in the specific embodiments of this invention. Although not wanting to be bound by theory, the optimization of plasmid DNA vectors for cardiac and muscle mediated plasmid mediated gene supplementation will increase their utility for delivery of therapeutic proteins including anti-coagulation factors, superoxide dismutase (“SOD”), hemoxygenase or other therapeutic molecules.

Construction of synthetic promoter and reporter plasmids. A 144 bp EcoRI/EagI fragment of chicken skeletal α-actin promoter (Chow et al., 1991; Lee et al., 1994) which contains the TATA box at −25bp upstream the cap site, a Sp1 pair in between −35 and −65bp and a TEF-1 site at −65bp, was removed from plasmid p612aACATMLC (Chow and Schwartz, 1990). The fragment was cloned into the EcoRI/EagI sites of pBluescript KS+ to generate pBS-SK144. pBS-SK144 was then cut SacI/HindIII, and the SK144 fragment, now with appropriate cloning sites was moved into the SacI/HindIII sites of pGL-2 basic vector (Promega, Madison, Wis., USA) to generate pSK144GL-2. All synthetic fragments had EagI cohesive ends and were cloned into EagI site of pSK144GL-2, to create synthetic promoter constructs driving luciferase. The pSK448GL-2 was utilized as a muscle specific control that contained a 448 bp chicken skeletal α-actin promoter (Draghia-Akli et al., 1997) cloned into the SacI/HindIII sites of the same pGL-2 basic vector. Additional methods for the construction of synthetic promotes and reporter plasmids are described in U.S. Pat. No. 6,410,228 (“the '228 Patent), issued on Jun. 25, 2002 and entitled “Method for the Identification of Synthetic Cell- or Tissue Specific Transcriptional Regulatory Regions” with Schwartz et al., listed as inventors, the entire content of which is hereby incorporated by reference.

Two complementary oligonucleotides were synthesized for each individual control element, phosphorylated and annealed to yield short DNA fragments. The oligonucleotide sequences were as follows:

| | | |
|---|---|---|
| SRE | 5′-GACACCCAAATATGGCGACGG-3′ | (SeqID#1) |
| | 3′-CTGTGGGTTTATACCGCTGCC-5′ | |
| MEF-1 | 5′-CCAACACCTGCTGCCTGCC-3′ | (SeqID#2) |
| | 3′-GGTTGTGGACGACGGACGG-5′ | |
| MEF-2 | 5′-CGCTCTAAAAATAACTCCC-3′ | (SeqID#3) |
| | 3′-GCGAGATTTTTATTGAGGG-5′ | |
| TEF-1 | 5′-CACCATTCCTCAC-3′ | (SeqID#4) |
| | 3′-GTGGTAAGGAGTG-5′ | |

The phosphorylation/annealing reaction was performed in a total volume of 300 μl in TEN buffer (10 mM Tris-HCl, pH 7.5; 1 mM EDTA, 50 mM NaCl) using sense and antisens strand oligonucleotides (20 μM each, equivalent to a total of 600 pmoles), 1 mM ATP and 0.5 U/ml of T4 polynucleotide kinase by heating to 70° C. for 15 minutes and cooling down to room temperature over 30 minutes.

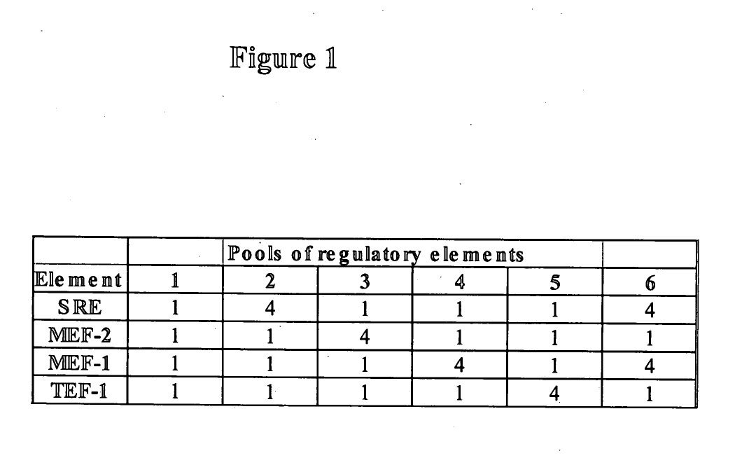

Different combination of SRE, MEF-1, MEF-2 and TEF-1 were then ligated in a total volume of 1 00 μl using different molar ratio (FIG. 1), maintaining a constant total amount of oligonucleotide of 200 pmoles. The core motif of each regulatory element (underlined) was flanked by adjacent sequence so that the binding sites of the regulatory elements would face the same side of the DNA helix when assembled together. The ligation reaction was completed with T4 ligase in 150 μl. After ligation, the combination of elements was run on a 6% acrylamide gel. The 75-300-bp region was cut and eluted in 2 volumes of diffusion buffer at 37° C. overnight. The DNA was extracted using Qiaex II Gel Extraction Kit (Qiagen Inc., Chatsworth, Calif., USA) and incubated in 150 μl with phosphorylated and annealed Sp1 element (2.5 nmoles) and 10 U of T4 ligase at 16° C. overnight. Since each of the Sp1 elements (′5-CCGTCCGCCCTCGG-3′) contains EagI half at both ends, an intact EagI restriction site was generated wherever two Sp1 elements were ligated together. The reaction was cleaned up (Qiaquick Nucleotide Removal Kit), digested with EagI and cloned into the EagI site of SK144GL-2 luciferase reporter construct, which resulted in a library of randomized synthetic-promoter-recombinants that were operatively linked to a reporter gene. The clones that gave the best results in the transfection studies were sequenced automatically.

Amplification and selection of the randomized synthetic-promoter-recombinant clones. The entire library of randomized synthetic-promoter-recombinants was transformed and then amplified in E. coli DH5α cells, plated on agar growth medium, and individual specific clones screened by transfection into muscle cells. The clones that gave the best results in the transfection studies were then sequenced automatically.

The nucleic acid sequences reported herein are believed to be correct, however a small percentage of sequence errors may be present. One skilled in the art could readily obtain the correct synthetic regulatory region by identifying the particular elements and their positions in the region from the sequence provided, and constructing the synthetic regulatory regions from those elements in the same positions and orientations.

Screening for high transcriptional activity synthetic promoters. Miniprep DNA was used for transfection during the initial screening of synthetic promoters. After plating 4000 cells/well in 96 well dishes, cells were transfected with 15 ng plasmid/well using lipofectamine and collected 72 h post-transfection, using the conditions described in the next paragraph.

Cell culture. Minimal Essential Medium (MEM), heat inactivated horse serum (“HIHS”), gentamycin, Hanks Balanced Salt Solution (HBSS), lipofectamine were obtained from Gibco BRL (Grand Island, N.Y.). Primary chicken myoblast and mouse cardiac cultures were obtained as described (Bergsma et al., 1986). Cells were plated 24 h prior to transfection at a density of 1.5 million cells/100 mm plate, in MEM supplemented with 10% HIHS, 5% chicken embryo extract (CEE) and gentamycin. Cells were maintained in a humidified 5% CO₂ 95% air atmosphere at 37° C. Cells were transfected with 4 μg of plasmid per 100 mm plate, using lipofectamine, according to the manufacturer instructions. After transfection, the medium was changed to MEM which contained 2% HIHS, 2% CEE for at least 24 h to allow the cells to differentiate. Media and cells were harvested 24,48,72 and 96 h post-differentiation. The samples and controls were assayed in quadruplicate in at least two different rounds of transfection. The efficiency of transfection was estimated by β-galactosidase histochemistry of control plates to be 10%. The cells were homogenized in Promega reporter lysis buffer for luciferase, beta-galactosidase and protein assays.

Northern blot analysis. 10-20 μg of total RNA was DNase I treated (Gibco BRL), size separated in 1.5% agarose-formaldehyde gel and transferred to Gene Screen nylon membrane (DuPont Research Products, Boston, Mass.). The membranes were hybridized with cDNA probes ³²P labeled by random priming (Ready-to-Go DNA labeling kit, Pharmacia Biotech, Piscataway, N.J.). Hybridization was carried out at 45° C. in a solution which contained 50% formamide, 5×SSPE, 5×Denhardts, 1% SDS, 200 μg/ml sheared salmon sperm DNA. Membranes were washed twice for 10 minutes in 2×SSPE/1%SDS at room temperature and twice for 30 minutes in 0.2×SSPE/1%SDS at 68° C. Blots were subsequently exposed to X-ray film (Kodak X-Omat AR; Eastman Kodak, Rochester, N.Y.) at −80° C. with intensifying screens.

Transgenic animals study. Transgenic mice carrying E.coli beta-galactosidase (“β-gal”) with an NLS under the control of the SPc5-12 promoter were generated by standard oocyte injection. Three different lines of 5 weeks old SPc5-12β-gal mice and control littermates were killed and samples of different organs and skeletal muscles were collected, stored at −80° C. For β-gal histochemistry, tissues were sectioned at 10 μm, fixed and stained.

Intramuscular injection of plasmid DNA in adult mice. Plasmid preparation of SPc5-12 and SK448 were diluted in PBS pH=7.4 to 1 mg/ml. ICR male mice (Harlem Laboratories, Houston, Tex.) were anesthetized with 0.5 ml/kg of a combination of ketamine (42.8 mg/ml), xylazine (8.2 mg/ml) and acepromazine (0.7 mg/ml). Fifty micrograms of plasmid in 25 μl sterile PBS was injected directly into the anterior tibialis of mice. At 1, 2 and 4 weeks after the injection, the injected muscle was snap frozen in liquid nitrogen. Muscles were homogenized in PBS, pH=7.4 containing 0.2% Triton x-100 and protease inhibitors: leupeptin, 0.7 μg/ml, pepstatin 10 μg/ml and aprotinin 2 μg/ml (Boehringer Mannheim, Indianapolis, Ind.). Muscle extracts were centrifuged at 10,000×g for 30 minutes at 4° C. and the supernatant recovered. Protein assays were performed using Bio-Rad Protein Assay (Bio-Rad Laboratories, Hercules, Calif.) and luciferase and β-galactosidase activity was measured. At each time point, 6-15 animals were used for each construct. The experiments were repeated twice.

Mouse growth hormone RIA. Mouse GH in plasma was measured with a heterologous rat assay system (Amersham, Arlington Heights, Ill.). The sensitivity of the assay was 0.16 ng/tube. The intra- and interassay coefficients of variation were 6.5 and 6.8% respectively.

Statistics. Data were analyzed using Microsoft Excel statistics analysis package. Values shown in the figures are the mean±s.e.m. to exert the desired effect.

The invention may be better understood with reference to the following examples, which are representative of some of the embodiments of the invention, and are not intended to limit the invention.

### EXAMPLE 1

Construction of synthetic promoter libraries. Although not wanting to be bound by theory, the endogenous promoter of skeletal α-actin is considered a very strong promoter. For example, when poly-A mRNA is isolated from an adult avian muscle, approximately 9% of the total poly-A mRNA isolated comprises skeletal α-actin mRNA, which is the highest expressed level of any poly-A mRNA species in cardiac or skeletal muscle (Schwartz and Rothblum, 1981). A short core fragment (i.e. SK144) of the chicken skeletal α-actin promoter was used as the minimal sequence to insert synthetic regulatory elements (Lee et al., 1994),(Chow et al., 1991). The core motif of each regulatory element was flanked by adjacent sequences that are conserved in the natural genes to allow the regulatory elements to anneal on the same face of the DNA helix. For example the serum regulatory element (“SRE”) sequence corresponds to the proximal SK SRE1, GCTGC motif adjacent to the MEF-1 is conserved in the muscle creatine kinase gene and rat myosin light chain gene. Different combinations of SRE, MEF-1, MEF-2 and TEF-1 oligonucleotide (FIG. 1) were annealed and then capped by ligation with Sp1 elements, since Sp1 has been shown to act in synergy with SREs and E-boxes. It has also been shown that Sp1 binding sites are essential for de novo methylation protection of CpG islands and non-island DNA regions (Machon et al., 1998). Synthetic promoter libraries were generated from DNA fragments containing about 5-20 regulatory elements and ligated into a minimal actin-reporter plasmid that expresses the luciferase reporter gene.

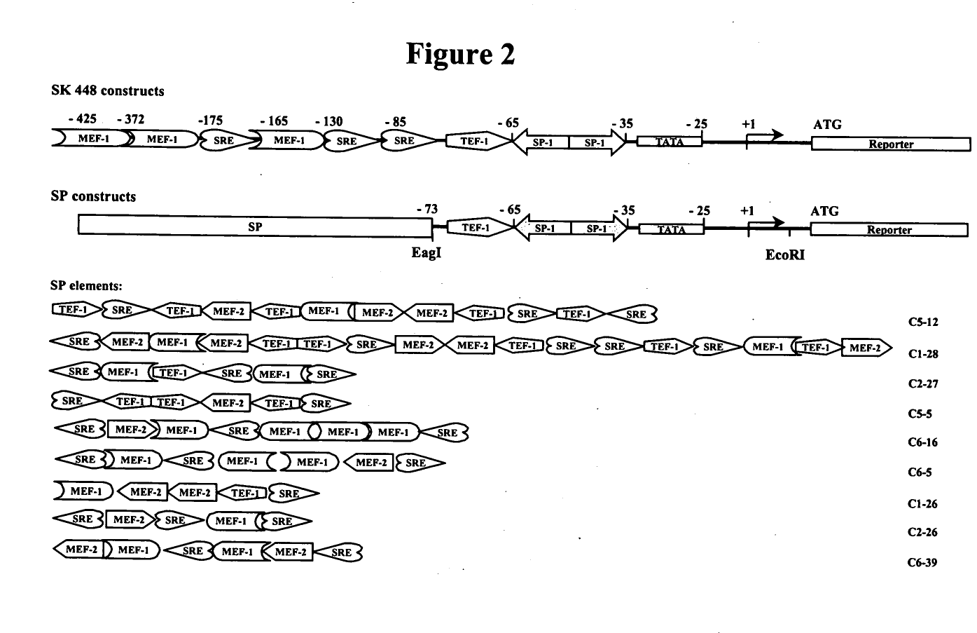

Screening synthetic promoters with high transcriptional activity. The in vitro luciferase activity was measured in more than 1000 different clones in 96 well dishes containing transiently transfected chicken primary myoblasts to determine the strength of the newly constructed synthetic promoters. A 448bp promoter fragment (−424/+24) (“SK448”) of the avian skeletal α-actin gene was used as a specific expression control in cardiac and skeletal muscle (FIG. 2). The SK448 promoter control has been shown to be active in differentiated skeletal muscle cells, but not in myoblasts (Bergsma et al., 1986; Chow and Schwartz, 1990; Lee et al., 1994). Cytomegalovirus (“CMV”) basic promoter was also used as a ubiquitous promoter control. Newly generated synthetic promoters, CMV promoters, and SK448 promoters were inserted into reporter construct plasmids and transfected into cells then placed into differentiation media for up to 72 hours to initiate withdrawal from the cell cycle and to induce post-fusion differentiation and muscle-specific promoter activation. At the end of this period the cells were harvested and assayed for the reporter gene activity.

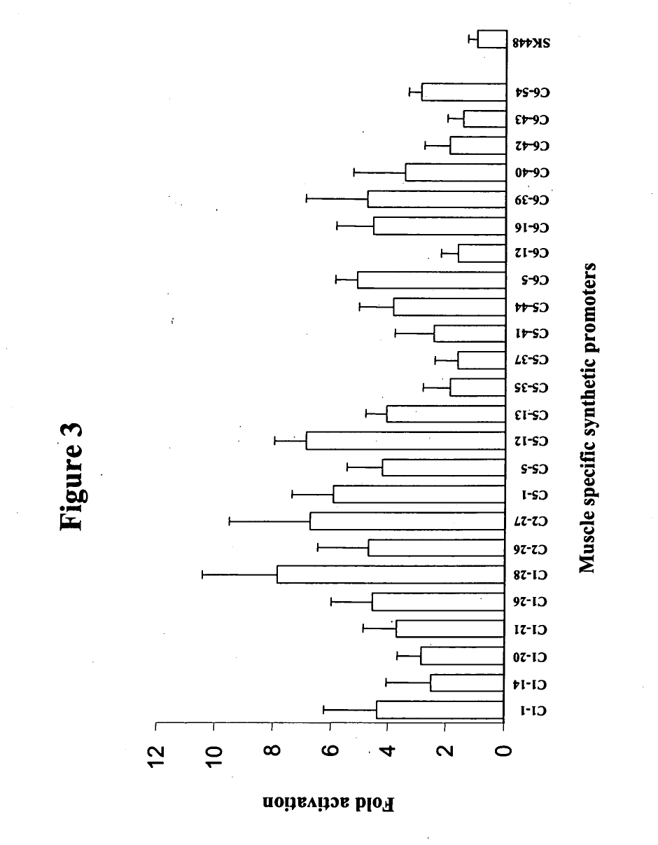

Promoters consisting of only multimerized single elements such as SREs, E-boxes, MEF-2 or TEF-1 had activities several-fold lower than the skeletal α-actin promoter 448 (data not shown). We observed that some promoters containing a combinatory pool of elements provided a 2 to 10 fold higher luciferase reporter gene activity (FIG. 3) when compared to SK448. Clones that displayed transcriptional activity greater than 2 times that of SK448 activity were examined further. Some clones from the first and fifth combinatorial pools, such as c1 -28, c5-12, c5-1, c5-5, where SRE, MEF-2, MEF- 1, TEF-1 were mixed in the ratio 1:1:1:1 and 1:1:1:4, respectively, had the highest in vitro and/or in vivo activity (see also FIG. 1).

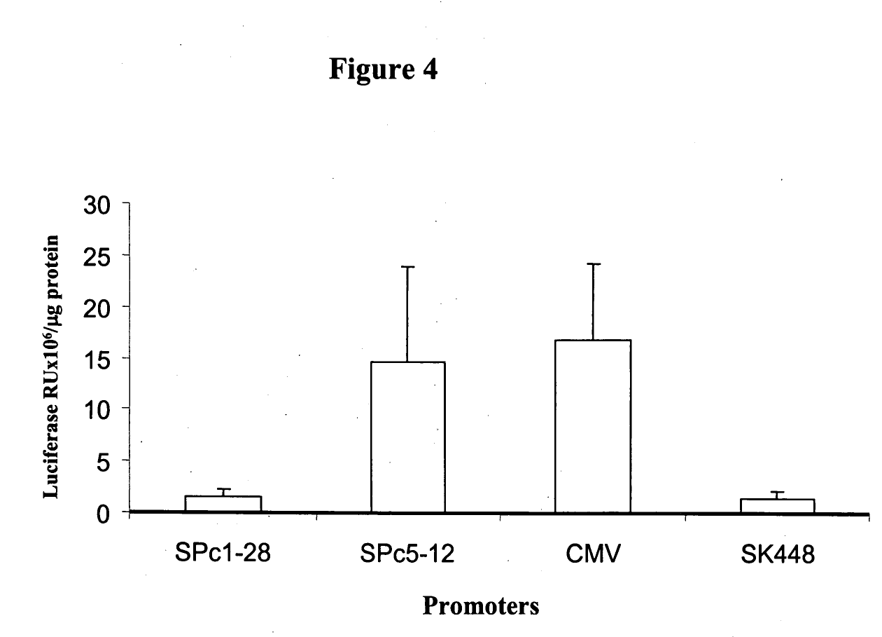

Cell culture assay systems cannot readily substitute for in vivo testing into the skeletal muscle of new plasmid constructs, as it has been shown that some muscle specific regulatory elements with high in vitro expression have less activity in vivo (Barnhart et al., 1998). Fifty micrograms of the most potent synthetic promoters (SPc1-28 and SPc5-12), SK448 and CMV plasmids were injected into the tibialis anterior muscle of adult ICR mice (n=6/group). One week later (FIG. 4), the activity of CMV and SPc5-12 was similar (16.77±7.43 and 14.59±9.39×10⁶RU/μg protein, respectively), while SK448 and SPc1-28 were 10 fold less active (1.44±0.76 and 1.58±0.65×10⁶RU/μg protein, respectively). SPc5-12 was then chosen for further studies.

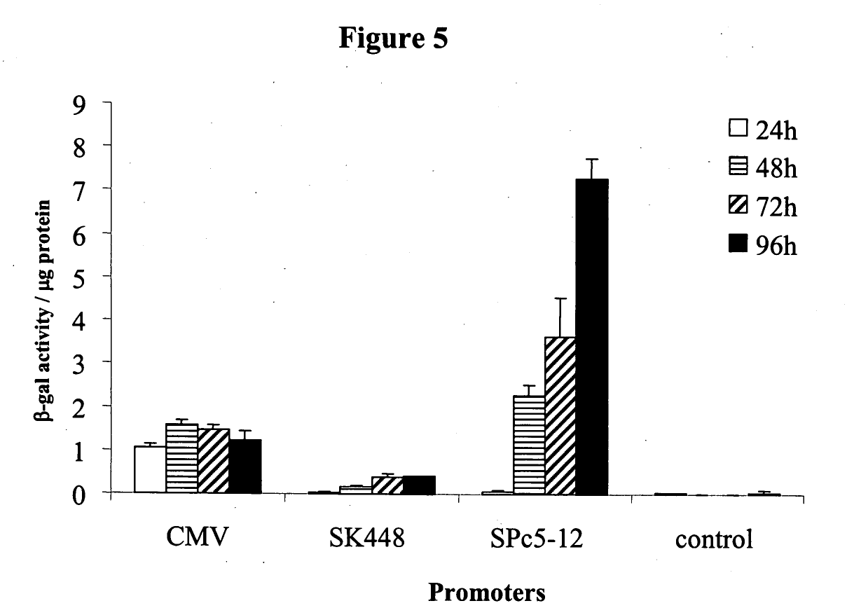

SPc5-12 was tested over a 96 hour time-course during primary avian muscle cell myogenesis in culture where replicating myoblasts withdraw from the cell cycle, fuse and form multinucleated terminally differentiated myotubes (FIG. 5). CMV promoter was active in both myoblasts and myotubes at similar levels (1.05±0.06×10⁶RU (relative units)/μg protein at 24 h, 1.22±0.22×10⁶RU/μg protein at 96 h). SK448 expression increased only after 48 hours (0.17±0.016×10⁶ RU/μg protein at 48 h, 0.37±0.09×10⁶ RU/μg protein at 72 h, 0.41±0.06×10⁶ RU/μg protein at 96 h), which correspond to the pattern of activation of SK promoters, active in myotubes but not in replicating myoblasts (Hayward and Schwartz, 1986). SPc5-12 mimicked the pattern of activation of SK448. However, SPc5-12 was 10 fold more active than SK448 and 2-6 fold higher than CMV promoter at 96 h (2.27±0.23×10⁶ RU/μg protein at 48 h, 3.62±0.91×10⁶ RU/μg protein at 72 h and 7.25±0.48×10⁶ RU/μg protein at 96 h).

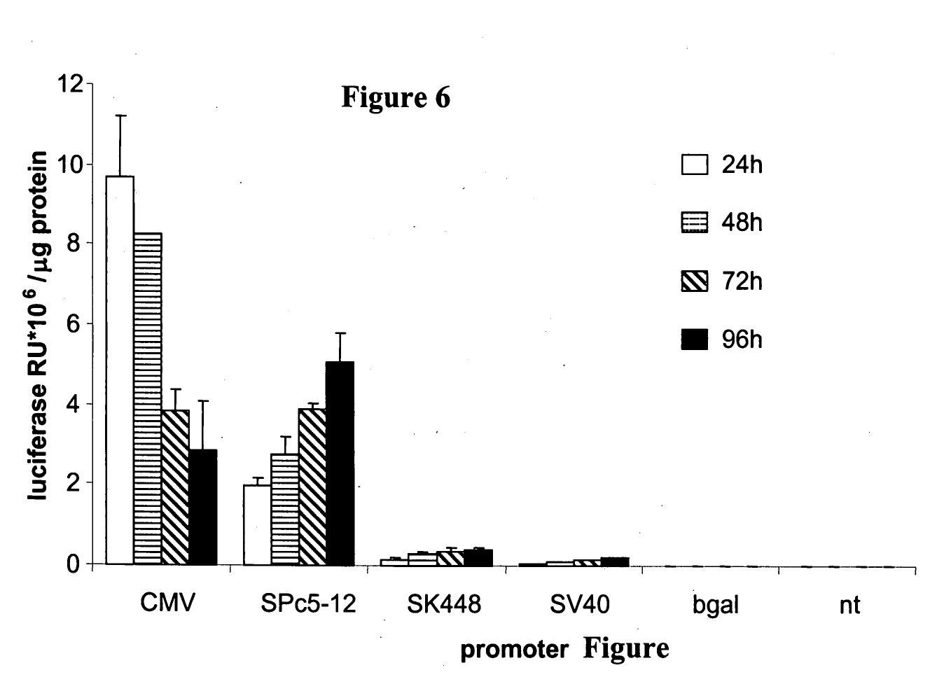

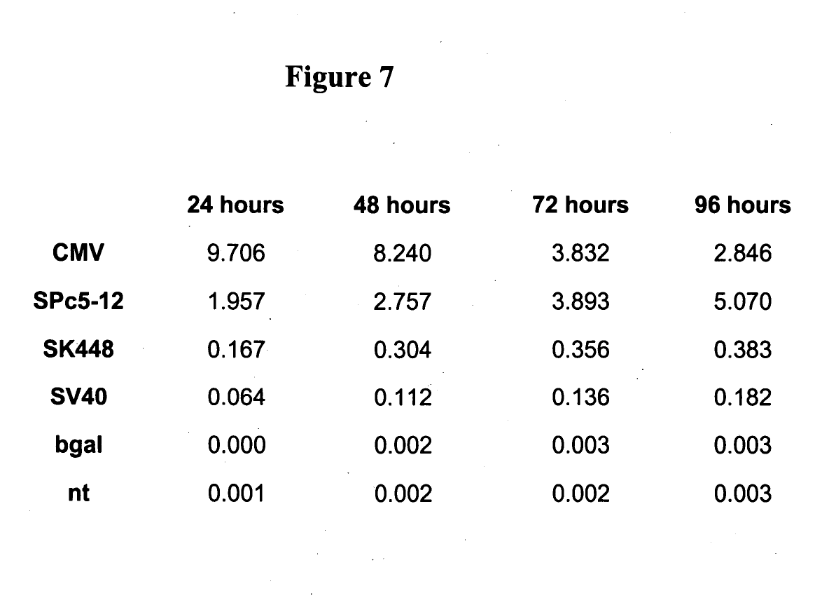

SPc5-12 was tested in primary cardiac myocytes over a 96-hour time course (FIG. 6), and compared with the ubiquitous promoters CMV and SV40 and with the muscle specific promoter SK448. As shown, CMV promoter has high initial activity in cardiac cells, which decreases over time. SK448 and SPc5-12 activities increase during the same time period, with long-term activation and higher activity than the baseline. Similarly to the skeletal muscle cells, in cardiac cells at 96 hour post-transfection, the SPc5-12 promoter has 13-fold higher expression than the naturally occurring SK448, and 2-fold higher activity than CMV (FIG. 7).

### EXAMPLE 4

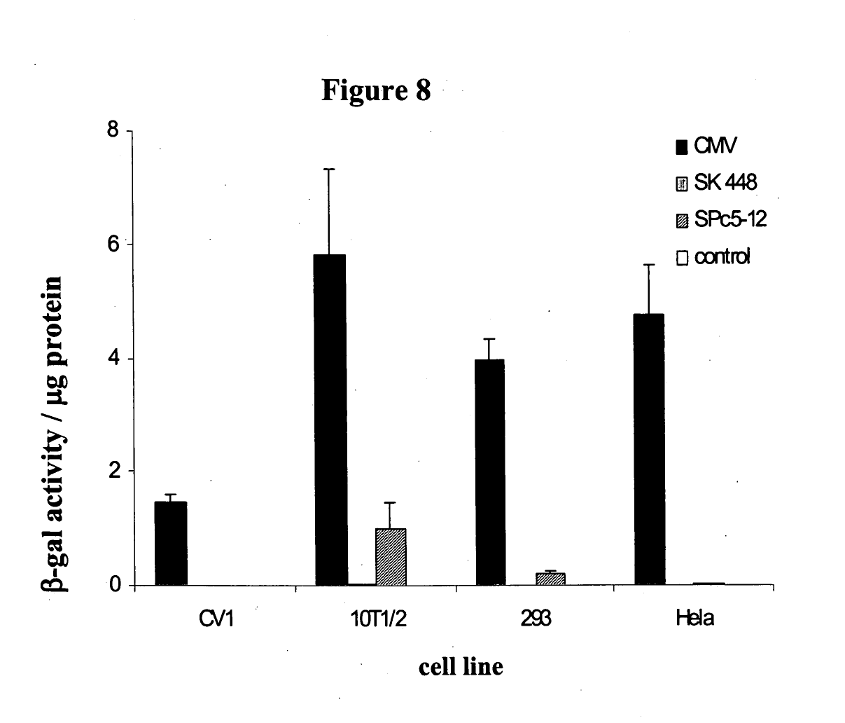

In vitro and in vivo specificity of SPc5-12 promoter. The specificity of SPc5-12 promoter was evaluated by transient transfections in several non-muscle cell lines. In the CV1 line (monkey kidney fibroblasts), HeLa cells (human cervix epitheloid carcinoma), 293 line (human transformed embryonic kidney) and 10 T1/2 line (mouse embryonic fibroblasts) specific β-gal activity of SPc5-12 and SK 448 constructs was relatively low compared with the prevalently expressed CMV promoter (FIG. 8).

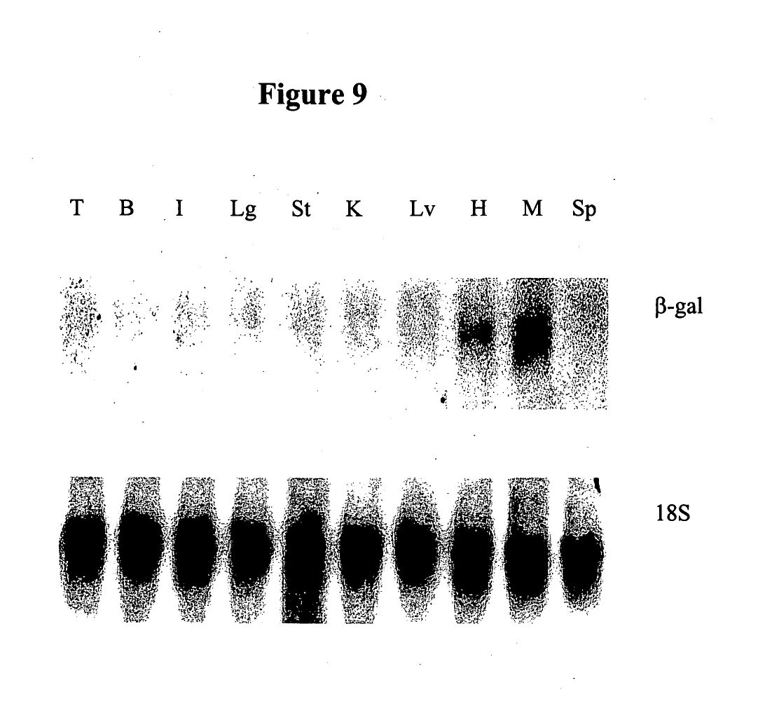

We then generated lines of transgenic mice carrying E.coli β-galactosidase (β-gal) with a nuclear localization signal (nls) under the control of the SPc5-12 promoter to determine its in vivo specificity. At the end of 5 weeks, several different SPc5- 12 transgenic β-gal mice were killed and samples of different organs (lung, liver, brain, spleen, intestine, stomach, kidney, testis) and heart and skeletal muscles were frozen in liquid nitrogen. β-gal tissue specific expression was evaluated by Northern blot analysis of total RNA (FIG. 9) and histochemistry techniques (data not shown). RNA blot analysis revealed β-gal transcripts only in muscle and heart samples in all positive lines of SPc5-12 transgenic mice; no expression was detected in non-myogenic organs. Histologically, β-gal positive nuclei were present in muscle fibers, as with the original SK448 promoter, but not in the control littermates. The pattern of expression was similar in 2 other transgenic lines.

### EXAMPLE 3

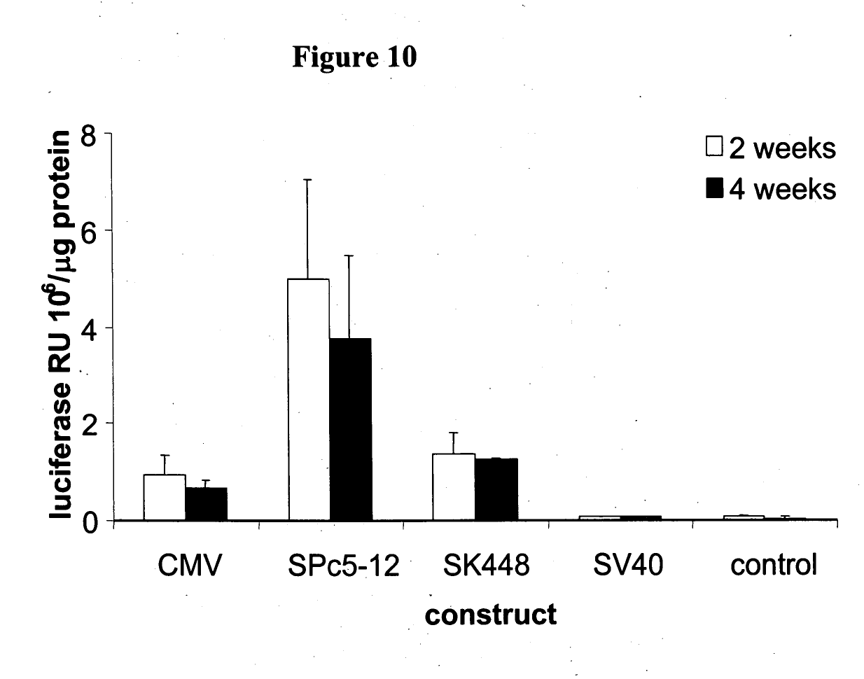

In vivo activity of SPc5-12 promoter. In vivo expression of SPc5-12 promoter was compared to that of the ubiquitous promoters CMV and SV40, and with the muscle specific SK448 promoter, after direct intra-muscular injection in adult immunocompetent mice (FIG. 10). At 2 and 4 weeks post-injection, the SPc5-12 driven construct had an activity 3-5 fold higher that that of the SK448 promoter (SPc5-12, 2 weeks: 4.97±2.07×10⁶ RU/μg protein, 4 weeks: 3.78±1.71×10⁶ RU/μg protein vs. SK448, 2 weeks: 1.37±0.43×10⁶ RU/μg protein, 4 weeks: 1.25±0.04×10⁶ RU/μg protein) and 6-8 time greater then that of the CMV promoter (2 weeks: 0.94±0.4×10⁶ RU/μg protein, 4 weeks: 0.65±0.16×10⁶ RU/μg protein). The SV40 construct was 100 fold less active at each of these time points (2 weeks: 0.05±0.02×10⁶ RU/μg protein, 4 weeks: 0.04±0.008×10⁶ RU/μg protein. These results show that in vivo transfection of the SPc5-12 into skeletal muscle results in significantly higher expression than conventional promoters do.

The ability of our synthetic promoter to ensure production of therapeutic levels of a secreted protein was determined. Human growth hormone releasing hormone (“hGHRH”) cDNA was cloned downstream of the SPc5-12 promoter. The same construct, but with a CMV promoter, was used as a positive control. Biologically active hGHRH secreted by the muscle cells stimulated the secretion of endogenous growth hormone (“mGH”) from the anterior pituitary of the injected mice. Seven days after direct intra-muscular injection of 30 micrograms of SPc5-12-GHRH plasmid in adult mice, serum mouse GH (“mGH”) was measured using a specific RIA. Serum mGH increased in both SPc5-12-GHRH and CMV-GHRH injected mice compared to control levels (24.84±13.15 ng/ml and 21.19±11.05 ng/ml, respectively vs. 1.7±0.1 ng/ml). The values obtained using these synthetic promoters (in a quantity of 30 μg of plasmid) were 1.5 fold higher than that obtained using 100 μg of pSK-GHRH in a previous study in our laboratory (Draghia-Akli et al., 1997), a five fold increase in activation when normalizing for the plasmid quantity.

The above synthetic promoters can be utilized for organ specific expression of various therapeutic genes in a mammalian host. One skilled in the art recognizes that the promoters described herein can direct the expression of any number of different genes that are useful for plasmid mediated gene supplementation. Methods and compositions for constructing promoters that can be utilized for effective gene transfer of an expression vector to a host cell in accordance with the present invention to a host cell can be monitored in terms of a therapeutic effect (e.g. alleviation of some symptom associated with the particular disease being treated) or, further, by evidence of the transferred gene or high expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

The above tissue specific synthetic promoters can be utilized in diverse vector constructs and administered to a mammalian host for various therapeutic effects. One skilled in the art recognizes that different methods of delivery may be utilized to administer a tissue specific synthetic expression vector into a cell. Examples include: (1) methods utilizing physical means, such as electroporation (electricity), a gene gun (physical force) or applying large volumes of a liquid (pressure); and (2) methods wherein the tissue specific synthetic expression vector is complexed to another entity, such as a liposome or transporter molecule.

Accordingly, the present invention provides a method of transferring a tissue specific therapeutic gene to a host, which comprises administering the vector of the present invention, preferably as part of a composition, using any of the aforementioned routes of administration or alternative routes known to those skilled in the art and appropriate for a particular application. Effective gene transfer of a tissue specific expression vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These compositions and methods described herein are by no means all-inclusive, and further methods to suit the specific application will be apparent to the ordinary skilled artisan. Moreover, the effective amount of the compositions can be further approximated through analogy to compounds known to exert the desired effect.

### Reference List

### U.S. Patent Documents

U.S. Pat. No. 6,410,228 filed on Jul. 14, 1998 and entitled “Method for the identification of synthetic cell- or Tissue-specific transcriptional regulatory regions” with Schwartz, et al. listed as inventors.

Other Publication List:

Acsadi, G., S. S. Jiao, A. Jani, D. Duke, P. Williams, W. Chong, and J. A. Wolff. 1991. Direct gene transfer and expression into rat heart in vivo. New Biologist 3:71-81.

Barnhart, K., J. Hartikka, M. Manthorpe, J. Norman, and Hobart P. 1998. Enhancer and promoter chimeras in plasmids designed for intramuscular injection: a comparative in vivo and in vitro study. American Society of Gene Therapy-1st Annual Meeting Abstract 303.

Bergsma, D. J., J. M. Grichnik, L. M. Gossett, and R. J. Schwartz. 1986. Delimitation and characterization of cis-acting DNA sequences required for the regulated expression and transcriptional control of the chicken skeletal alpha-actin gene. Molecular & Cellular Biology 6:2462-2475.

Buvoli, M., S. J. Langer, S. Bialik, and L. A. Leinwand. 2002. Potential limitations of transcription terminators used as transgene insulators in adenoviral vectors. Gene Ther. 9:227-231.

Carroll, S. L., D. J. Bergsma, and R. J. Schwartz. 1986. Structure and complete nucleotide sequence of the chicken alpha-smooth muscle (aortic) actin gene. An actin gene which produces multiple messenger RNAs. J. Biol. Chem. 261:8965-8976.

Chang, P. S., L. Li, J. McAnally, and E. N. Olson. 2001. Muscle specificity encoded by specific serum response factor-binding sites. J. Biol. Chem. 276:17206-17212.

Chow, K. L., M. E. Hogan, and R. J. Schwartz. 1991. Phased cis-acting promoter elements interact at short distances to direct avian skeletal alpha-actin gene transcription. Proc. Natl. Acad. Sci. USA 88:1301-1305.

Chow, K. L. and R. J. Schwartz. 1990. A combination of closely associated positive and negative cis-acting promoter elements regulates transcription of the skeletal alpha-actin gene. Molecular & Cellular Biology 10:528-538.

Condorelli, G., R. Roncarati, J. Ross, Jr., A. Pisani, G. Stassi, M. Todaro, S. Trocha, A. Drusco, Y. Gu, M. A. Russo, G. Frati, S. P. Jones, D. J. Lefer, C. Napoli, and C. M. Croce. 2001. Heart-targeted overexpression of caspase3 in mice increases infarct size and depresses cardiac function. Proc. Natl. Acad. Sci. U. S. A 98:9977-9982.

Ding, E., H. Hu, B. L. Hodges, F. Migone, D. Serra, F. Xu, Y. T. Chen, and A. Amalfitano. 2002. Efficacy of gene therapy for a prototypical lysosomal storage disease (GSD-II) is critically dependent on vector dose, transgene promoter, and the tissues targeted for vector transduction. Mol. Ther. 5:436-446.

Draghia-Akli, R., M. L. Fiorotto, L. A. Hill, P. B. Malone, D. R. Deaver, and R. J. Schwartz. 1999. Myogenic expression of an injectable protease-resistant growth hormone-releasing hormone augments long-term growth in pigs. Nat. Biotechnol. 17:1179-1183.

Draghia-Akli, R., X. G. Li, and R. J. Schwartz. 1997. Enhanced growth by ectopic expression of growth hormone releasing hormone using an injectable myogenic vector nature biotechnology 15:1285-1289.

Franz, W. M., T. Rothmann, N. Frey, and H. A. Katus. 1997. Analysis oftissue-specific gene delivery by recombinant adenoviruses containing cardiac-specific promoters. Cardiovasc. Res. 35:560-566.

Gossett, L. A., D. J. Kelvin, E. A. Sternberg, and E. N. Olson. 1989. A new myocyte-specific enhancer-binding factor that recognizes a conserved element associated with multiple muscle-specific genes. Molecular & Cellular Biology 9:5022-5033.

Hartikka, J., M. Sawdey, F. Cornefert-Jensen, M. Margalith, K. Barnhart, M. Nolasco, H. L. Vahlsing, J. Meek, M. Marquet, P. Hobart, J. Norman, and M. Manthorpe. 1996. An improved plasmid DNA expression vector for direct injection into skeletal muscle. Human Gene Therapy 7:1205-1217.

Hayward, L. J. and R. J. Schwartz. 1986. Sequential expression of chicken actin genes during myogenesis. Journal of Cell Biology 102:1485-1493.

Kelly, K. K., S. M. Meadows, and R. M. Cripps. 2002. Drosophila MEF2 is a direct regulator of Actin57B transcription in cardiac, skeletal, and visceral muscle lineages. Mech. Dev. 110:39-50.

Keogh, M. C., D. Chen, J. F. Schmitt, U. Dennehy, V. V. Kakkar, and N. R. Lemoine. 1999. Design of a muscle cell-specific expression vector utilising human vascular smooth muscle alpha-actin regulatory elements. Gene Ther. 6:616-628.

Larkin, S. B., I. K. Farrance, and C. P. Ordahl. 1996. Flanking sequences modulate the cell specificity of M-CAT elements. Molecular & Cellular Biology 16:3742-3755.

Lassar, A. B., R. L. Davis, W. E. Wright, T. Kadesch, C. Murre, A. Voronova, D. Baltimore, and H. Weintraub. 1991. Functional activity of myogenic HLH proteins requires hetero-oligomerization with E12/E47-like proteins in vivo. Cell 66:305-315.

Lee, T. C., Y. Shi, and R. J. Schwartz. 1992. Displacement of BrdUrd-induced YY1 by serum response factor activates skeletal alpha-actin transcription in embryonic myoblasts. Proc. Natl. Acad. Sci. USA 89:9814-9818.

Lee, T. C., Y. Zhang, and R. J. Schwartz. 1994. Bifunctional transcriptional properties of YY1 in regulating muscle actin and c-myc gene expression during myogenesis. Oncogene 9:1047-1052.

Li, X., E. M. Eastman, R. J. Schwartz, and R. Draghia-Akli. 1999. Synthetic muscle promoters: activities exceeding naturally occurring regulatory sequences. nature biotechnology 17:241-245.

Lin, H., M. S. Parmacek, G. Morle, S. Bolling, and J. M. Leiden. 1990. Expression of recombinant genes in myocardium in vivo after direct injection of DNA. Circulation 82:2217-2221.

Machon, O., J. Svoboda, J. Geryk, J. Hejnar, and V. Strmen. 1998. Sp1 binding sites inserted into the rous sarcoma virus long terminal repeat enhance LTR-driven gene expression. Gene 208 (1):73-82.

Mazda, 0. 2002. Improvement of nonviral gene therapy by Epstein-Barr virus (EBV)-based plasmid vectors. Curr. Gene Ther. 2:379-392.

Minty, A. and L. Kedes. 1986. Upstream regions of the human cardiac actin gene that modulate its transcription in muscle cells: presence of an evolutionarily conserved repeated motif. Molecular & Cellular Biology 6:2125-2136.

Montgomery, D. L., J. B. Ulmer, J. J. Donnelly, M. A. Liu, Immunization, Antibodies, Cell-mediated immunit, Protective immunity, Foreign-gene expression, and Plasmid vectors. 1997. DNA vaccines. Pharmacology & Therapeutics 74:195-205.

Nemer, G. and M. Nemer. 2001. Regulation of heart development and fimction through combinatorial interactions of transcription factors. Ann. Med. 33:604-610.

O'Connell, T. D., D. G. Rokosh, and P. C. Simpson. 2001. Cloning and characterization of the mouse alpha1C/A-adrenergic receptor gene and analysis of an alpha1C promoter in cardiac myocytes: role of an MCAT element that binds transcriptional enhancer factor-1 (TEF-1). Mol. Pharmacol. 59:1225-1234.

Olson, E. N., T. J. Brennan, T. Chakraborty, T. C. Cheng, P. Cserjesi, Edmondson, G. James, and L. Li. 1991. Molecular control of myogenesis: antagonism between growth and differentiation. Molecular & Cellular Biochemistry 104:7-13.

Phillips, M. I., Y. Tang, K. Schmidt-Ott, K. Qian, and S. Kagiyama. 2002. Vigilant vector: heart-specific promoter in an adeno-associated virus vector for cardioprotection. Hypertension 39:651-655.

Prentice, H., R. A. Kloner, Y. Li, L. Newman, and L. Kedes. 1996. Ischemic/reperfused myocardium can express recombinant protein following direct DNA or retroviral injection. J. Mol. Cell Cardiol. 28:133-140.

Roell, W., Y. Fan, Y. Xia, E. Stoecker, P. Sasse, E. Kolossov, W. Bloch, H. Metzner, C. Schmitz, K. Addicks, J. Hescheler, A. Welz, and B. K. Fleischmann. 2002. Cellular cardiomyoplasty in a transgenic mouse model. Transplantation 73:462-465.

Rothermel, B. A., T. A. McKinsey, R. B. Vega, R. L. Nicol, P. Mammen, J. Yang, C. L. Antos, J. M. Shelton, R. Bassel-Duby, E. N. Olson, and R. S. Williams. 2001. Myocyte-enriched calcineurin-interacting protein, MCIP1, inhibits cardiac hypertrophy in vivo. Proc. Natl. Acad. Sci. U. S. A 98:3328-3333.

Schwartz, R. J. and K. N. Rothblum. 1981. Gene switching in myogenesis: differential expression of the chicken actin multigene family. Biochemistry 20:4122-4129.

Skarli, M., A. Kiri, G. Vrbova, C. A. Lee, and G. Goldspink. 1998. Myosin regulatory elements as vectors for gene transfer by intramuscular injection. Gene Therapy 5:514-520.

Stewart, A. F., S. B. Larkin, I. K. Farrance, J. H. Mar, D. E. Hall, and C. P. Ordahl. 1994. Muscle-enriched TEF-1 isoforms bind M-CAT elements from muscle-specific promoters and differentially activate transcription. J. Biol. Chem. 269:3147-3150.

Vale, P. R., D. W. Losordo, T. Tkebuchava, D. Chen, C. E. Milliken, and J. M. Isner. 1999. Catheter-based myocardial gene transfer utilizing nonfluoroscopic electromechanical left ventricular mapping. J. Am. Coll. Cardiol. 34:246-254.

Wang, D., P. S. Chang, Z. Wang, L. Sutherland, J. A. Richardson, E. Small, P. A. Krieg, and E. N. Olson. 2001. Activation of cardiac gene expression by myocardin, a transcriptional cofactor for serum response factor. Cell 105:851-862.

Webster, K. A. and N. H. Bishopric. 2000. Molecular aspects and gene therapy prospects for diastolic failure. Cardiol. Clin. 18:621-635.

Weintraub, H., R. Davis, D. Lockshon, and A. Lassar. 1990. MyoD binds cooperatively to two sites in a target enhancer sequence: occupancy of two sites is required for activation. Proc. Natl. Acad. Sci. USA 87:5623-5627.

Wolff, J. A., J. J. Ludtke, G. Acsadi, P. Williams, and A. Jani. 1992. Long-term persistence of plasmid DNA and foreign gene expression in mouse muscle. Human Molecular Genetics 1:363-369.

Xu, Z. L., H. Mizuguchi, A. Ishii-Watabe, E. Uchida, T. Mayumi, and T. Hayakawa. 2002.

Strength evaluation of transcriptional regulatory elements for tansgene expression by adenovirus vector. J. Control Release 81:155-163.

Zhang, X., J. Chai, G. Azhar, P. Sheridan, A. M. Borras, M. C. Furr, K. Khrapko, J. Lawitts, R. P. Misra, and J. Y. Wei. 2001. Early postnatal cardiac changes and premature death in transgenic mice overexpressing a mutant form of serum response factor. J. Biol. Chem. 276:40033-40040.

What is claimed is:

1. A cardiac specific-synthetic promoter produced by a method comprising:
(a) introducing a library of randomized synthetic-promoter-recombinant expression constructs into a first-population of cells forming a first-test-population of cells;
(b) screening the first-test-population of cells for a first cardiac-specific-clone having a first-transcriptional activity that is higher than a control-transcriptional activity; and
(c) utilizing the cardiac specific-synthetic promoter from the first-cardiac-specific clone as the cardiac specific-synthetic promoter for a cardiac-specific-synthetic expression construct;
wherein,
each of the randomized synthetic-promoter-recombinant expression constructs are operatively linked to a reporter gene to form a nucleic acid expression construct; and
the control-cardiac-specific-clone comprises a known-promoter operatively linked to the reporter gene forming a control-nucleic acid expression construct having the control-transcriptional activity in the first- population of cells.

2. The cardiac specific-synthetic promoter of claim 1, wherein the first-population of cells comprise cells in vitro.

3. The cardiac specific-synthetic promoter of claim 1, further comprising:
second-screening the first cardiac-specific-clone in a second-test-population of cells before utilizing the cardiac-specific-synthetic promoter as the cardiac-specific-synthetic promoter for the cardiac-specific-synthetic expression construct;
wherein,
the reporter gene from the first-cardiac-specific-clone having a second-transcriptional activity in the second-population of cells that is higher than a second-control-transcriptional activity of the control-cardiac-specific-clone introduced into the second-population of cells.

4. The cardiac specific-synthetic promoter claim 3, wherein the first-population of cells comprise cells in vitro, and the second-population of cells comprise cells in vivo.

5. The cardiac specific-synthetic promoter of claim 1, wherein cardiac specific synthetic promoter comprises c5-12 (SeqID#5).

6. The cardiac specific-synthetic promoter of claim 1, wherein cardiac specific synthetic promoter comprises c1-26 (SeqID#16); c2-26 (SeqID#17); c2-27 (SeqID#18); c5-5 (SeqID#19); c6-5 (SeqID#20); c6-16 (SeqID#21); or c6-39 (SeqID#22).

7. The cardiac specific-synthetic promoter of claim 1, wherein the cardiac-specific-synthetic promoter comprises a first-combination of cis-acting regulatory elements;
the first combination of cis-acting regulatory elements being selected from library of randomized synthetic-promoter-recombinants; and
the cardiac-specific synthetic promoter driving a transcriptional activity of the expressible gene in a population of cells that is higher than the transcriptional activity of the expressible gene driven by a control-promoter in the population of cells.

8. The cardiac specific-synthetic promoter of claim 7, wherein the cis-acting regulatory elements comprise SRE (SeqID#1); MEF-1 (SeqID#2); MEF-2 (SeqID#3); and TEF-1 (SeqID#4).

9. A method of using a cardiac specific-synthetic expression construct for expressing a gene in a cardiac cell comprising:
delivering into the cardiac cell a cardiac specific-synthetic expression construct;
wherein, the cardiac-specific-synthetic expression construct comprises a cardiac-specific-synthetic-promoter operatively-linked to an expressible gene.

10. The method of claim 9, wherein cardiac specific synthetic promoter comprises c5-12 (SeqID#5).

11. The method of claim 9, wherein cardiac specific synthetic promoter comprises c1-26 (SeqID#16); c2-26 (SeqID#17); c2-27 (SeqID#18); c5-5 (SeqID#19); c6-5 (SeqID#20); c6-16 (SeqID#21); or c6-39 (SeqID#22).

12. The method of claim 9, wherein the cardiac-specific-synthetic promoter comprises a first-combination of cis-acting regulatory elements;
the first combination of cis-acting regulatory elements being selected from library of randomized synthetic-promoter-recombinants; and
the cardiac-specific synthetic promoter driving a transcriptional activity of the expressible gene in a population of cells that is higher than the transcriptional activity of the expressible gene driven by a control-promoter in the population of cells.

13. The method of claim 12, wherein the cis-acting regulatory elements comprise SRE (SeqID#1); MEF-1 (SeqID#2); MEF-2 (SeqID#3); and TEF-1 (SeqID#4).

14. The method of claim 9, wherein delivering into the cardiac cell the cardiac specific-synthetic expression construct is via electroporation.

15. The method of claim 9, wherein the expressible-gene comprises a nucleic acid sequence that encodes a growth-hormone-releasing-hormone (“GHRH”) or functional biological equivalent thereof.

16. The composition of claim 15, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

17. The method of claim 15, wherein the encoded GHRH or fuctional biological equivalent thereof is of formula (SEQID#6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

18. The method of claim 9, wherein the cardiac specific-synthetic expression construct comprises SeqID No: 7, SeqID No: 8, SeqID No: 9, SeqID No: 10, SeqID No: 11, SeqID No: 12, SeqID No: 13, SeqID No: 14, or SeqID No: 15.

19. A method of synthesizing a cardiac specific synthetic expression construct comprising:
(a) identifying a cardiac-specific promoter; and
(b) operatively-linking the cardiac-specific promoter to an expressible gene to form the cardiac specific synthetic expression construct;
wherein; the cardiac-specific-synthetic promoter comprises a first- combination of cis-acting regulatory elements; and
the expressible gene comprises a nucleic acid expression construct with or without an operable-linked promoter.

20. The method of claim 19, wherein cardiac specific synthetic promoter comprises c5- 12 (SeqID#5).

21. The method of claim 19, wherein cardiac specific synthetic promoter comprises c1-26 (SeqID#16); c2-26 (SeqID#17); c2-27 (SeqID#18); c5-5 (SeqID#19); c6-5 (SeqID#20); c6-16 (SeqID#21); or c6-39 (SeqID#22).

22. The method of claim 19, wherein the first combination of cis-acting regulatory elements comprise being selected from library of randomized synthetic-promoter-recombinants; and
the cardiac-specific synthetic promoter driving a transcriptional activity of the expressible gene in a population of cells that is higher than the transcriptional activity of the expressible gene driven by a control-promoter in the population of cells.

23. The method of claim 22, wherein the cis-acting regulatory elements comprise SRE (SeqID#1); MEF-1 (SeqID#2); MEF-2 (SeqID#3); and TEF-1 (SeqID#4).

24. The method of claim 19, wherein delivering into the cardiac cell the cardiac specific-synthetic expression construct is via electroporation.

25. The method of claim 19, wherein the expressible-gene comprises a nucleic acid sequence that encodes a growth-hormone-releasing-hormone (“GHRH”) or functional biological equivalent thereof.

26. The composition of claim 25, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

27. The method of claim 25, wherein the encoded GHRH or functional biological equivalent thereof is of formula (SEQID#6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

28. The method of claim 19, wherein the cardiac specific-synthetic expression construct comprises SeqID No: 7, SeqID No: 8, SeqID No: 9, SeqID No: 10, SeqID No: 11, SeqID No: 12, SeqID No: 13, SeqID No: 14, or SeqID No: 15.

29. A method of using a cardiac specific-synthetic expression construct for expressing a gene in a cardiac cell comprising:
delivering into the cardiac cell a cardiac specific-synthetic expression construct;
wherein, the cardiac-specific-synthetic expression construct comprises a cardiac-specific-synthetic-promoter (SeqID No: 5) operatively-linked to an expressible gene.

30. The method of claim 29, wherein the expressible-gene comprises a nucleic acid sequence that encodes a growth-hormone-releasing-hormone (“GHRH”) or functional biological equivalent thereof.

31. The method of claim 30, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

32. The method of claim 30, wherein the encoded GHRH or functional biological equivalent thereof is of formula (SEQID#6):
—X₋₁—X₂-DAIFTNSYRKVL-X₃-QLSARKLLQDI-X₋₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

33. The method of claim 29, wherein the cardiac specific-synthetic expression construct comprises SeqID No: 7, SeqID No: 8, SeqID No: 9, SeqID No: 10, SeqID No: 11, SeqID No: 12, SeqID No: 13, SeqID No: 14, or SeqID No: 15.

34. A method of using a cardiac specific-synthetic expression construct for expressing a gene in a cardiac cell comprising:
delivering into the cardiac cell a cardiac specific-synthetic expression construct;
wherein, the cardiac-specific-synthetic expression construct comprises a cardiac-specific-synthetic-promoter (SeqID No: 18) operatively-linked to an expressible gene.

35. The method of claim 34, wherein the expressible-gene comprises a nucleic acid sequence that encodes a growth-hormone-releasing-hormone (“GHRH”) or functional biological equivalent thereof.

36. The method of claim 35, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

37. The method of claim 35, wherein the encoded GHRH or functional biological equivalent thereof is of formula (SEQID#6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine or a combination thereof.

38. The method of claim 34, wherein the cardiac specific-synthetic expression construct comprises SeqID No: 7, SeqID No: 8, SeqID No: 9, SeqID No: 10, SeqID No: 11, SeqID No: 12, SeqID No: 13, SeqID No: 14, or SeqID No: 15.


SEQUENCE LISTING (22 sequences)

SEQ ID NO: 1 - DNA, 21 bp, artificial sequence
  SRE control elements used in the promoters.
gacacccaaatatggcgacgg

SEQ ID NO: 2 - DNA, 19 bp, artificial sequence
  MEF-1 control element used in the promoters
ccaacacctgctgcctgcc

SEQ ID NO: 3 - DNA, 19 bp, artificial sequence
  MEF-2 control element used in the promoters.
cgctctaaaaataactccc

SEQ ID NO: 4 - DNA, 13 bp, artificial sequence
  TEF-1 control element used in the promoters.
caccattcctcac

SEQ ID NO: 5 - DNA, 335 bp, artificial sequence
  Nucleic acid sequence of an eukaryotic promoter c5-12.
cggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctaga

SEQ ID NO: 6 - protein, 40 aa, artificial sequence
  This is the artificial sequence for GHRH (1-40)OH.
XXDAIFTNSYRKVLXQLSARKLLQDIXXRQQGERNQEQGA

SEQ ID NO: 7 - DNA, 3534 bp, artificial sequence
  Nucleic acid sequence for the HV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 8 - DNA, 3534 bp, artificial sequence
  Nucleic acid sequence for the TI-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatatcgatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 9 - DNA, 3534 bp, artificial sequence
  Nucleic acid sequence for the TV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 10 - DNA, 3534 bp, artificial sequence; identical to SEQ ID NO: 8
  Nucleic acid sequence for the 15/27/28 GHRH plasmid.

SEQ ID NO: 11 - DNA, 2710 bp, artificial sequence
  Vector with a mouse codon optimized GHRH analog sequence
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctctgggtgctctttgtgatcctcatcctcaccagcggcagccactgcagcctgcctcccagccctcccttcaggatgcagaggcacgtggacgccatcttcaccaccaactacaggaagctgctgagccagctgtacgccaggaaggtgatccaggacatcatgaacaagcagggcgagaggatccaggagcagagggccaggctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 12 - DNA, 2713 bp, artificial sequence
  Vector with a rat codon optimized GHRH analog sequence
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttcttcgtgctgctgaccctgaccagcggaagccactgcagcctgcctcccagccctcccttcagggtgcgccggcacgccgacgccatcttcaccagcagctacaggaggatcctgggccagctgtacgctaggaagctcctgcacgagatcatgaacaggcagcagggcgagaggaaccaggagcagaggagcaggttcaactgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 13 - DNA, 2704 bp, artificial sequence
  Vector with a bovine codon optimized GHRH analog sequence
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggctcccacggctccctgccctcccagcctctgcgcatccctcgctacgccgacgccatcttcaccaacagctaccgcaaggtgctcggccagctcagcgcccgcaagctcctgcaggacatcatgaaccggcagcagggcgagcgcaaccaggagcagggagcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 14 - DNA, 2704 bp, artificial sequence
  Vector with a ovine codon optimized GHRH analog sequence
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggaagccacggcagcctgcccagccagcccctgaggatccctaggtacgccgacgccatcttcaccaacagctacaggaagatcctgggccagctgagcgctaggaagctcctgcaggacatcatgaacaggcagcagggcgagaggaaccaggagcagggcgcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 15 - DNA, 2713 bp, artificial sequence
  Vector with a chicken codon optimized GHRH analog sequence
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttctttgtgctgctgaccctgacctccggaagccactgcagcctgccacccagcccacccttccgcgtcaggcgccacgccgacggcatcttcagcaaggcctaccgcaagctcctgggccagctgagcgcacgcaactacctgcacagcctgatggccaagcgcgtgggcagcggactgggagacgaggccgagcccctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 16 - DNA, 382 bp, artificial sequence
  This is the synthetic promoter c1-26.
ggcggccgagggcggcggggcaggcagcaggtgttggcaccattcctcaccgctctaaaaataactcccgtgaggaatggtgccgtcgccatatttgggtgtcgacacccaaatatggcgacgggtgaggaatggtgggcaggcagcaggtgttgggacacccaaatatggcgacggccaacacctgctgcctgccgggagttatttttagagcggggagttatttttagagcggtgaggaatggtggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 17 - DNA, 218 bp, artificial sequence
  This is the synthetic promoter sequence for c2-26.
cggccgtcgccatatttgggtgtccgctctaaaaataactcccgacacccaaatatggcgacggggcaggcagcaggtgttgggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 18 - DNA, 230 bp, artificial sequence
  This is the synthetic sequence for c2-27.
cggccgtcgccatatttgggtgtcggcaggcagcaggtgttggcaccattcctcacccgtcgccatatttgggtgtcggcaggcagcagtgttgggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 19 - DNA, 231 bp, artificial sequence
  This is the synthetic promoter for c5-5.
cggccgtccgccctcgggacacccaaatatggcgacgggtgaggaatggtgcaccattcctcacgggagttatttttagagcggtgaggaatggtggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 20 - DNA, 255 bp, artificial sequence
  This is the synthetic promter for c6-5.
cggccgtcgccatatttgggtgtcccaacacctgctgcctgccccgtcgccatatttggtgtcggcaggcagcaggtgttggccaacacctgctgcctgccgggagttatttttagagcggacacccaaatatggcgacggccggggccgcattcctgggggccgggcggtgctcccgcccgcctcgataaaaggctccggggccggcggcggcccacgagctacccggaggagcgggaggcgccaagctctaga

SEQ ID NO: 21 - DNA, 283 bp, artificial sequence
  This is the synthetic promoter for c6-16.
cggccgtcgccatatttgggtgtccgctctaaaaataactcccccaacacctgctgcctgccccgtcgccatatttgggtgtcggcaggcagcaggtgttggccaacacctgctgcctgccccaacacctgctgcctgccccgtcgccatatttggtgtccgccctcggccggggccgcattcctgggggccgggcggtgctcccgcccgcctcgataaaaggctccggggccggcggcggcccacgagctacccggaggagcgggaggcgccaagctctaga

SEQ ID NO: 22 - DNA, 263 bp, artificial sequence
  This is the synthetic promoter for c6-39.
cggccgtccgccctcgggggagttatttttagagcgccaacacctgctgcctgccccgtcgccatatttgggtgtcggcaggcagcaggtgttgggggagttatttttagagcgccgtcgccatatttgggtgtcccgagggcggacggccggggccgcattcctgggggccgggcggtgctcccgcccgcctcgataaaaggctccggggccggcggcggcccacgagctacccggaggagcgggaggcgccaagctctaga